(19) United States
(12) Patent Application Publication
(10) Pub. No.: US 2018/0188266 A1
(43) Pub. Date: Jul. 5, 2018

(54) Characterization of Type 1 Collagen Synthesis By Fluorescence Imaging and Related Drug Screening Technique

(71) Applicant: Florida State University Research Foundation, Inc., Tallahassee, FL (US)

(72) Inventor: Branko Stefanovic, Tallahassee, FL (US)

(73) Assignee: Florida State University Research Foundation, Inc., Tallahassee, FL (US)

(21) Appl. No.: 15/860,207

(22) Filed: Jan. 2, 2018

Related U.S. Application Data
(60) Provisional application No. 62/441,674, filed on Jan. 3, 2017.

(51) Int. Cl. G01N 33/68; G01N 33/58; G01N 33/50

(52) U.S. Cl. CPC G01N 33/6887; G01N 2333/78; G01N 33/5038; G01N 33/582

(57) ABSTRACT

A method includes fluorescence imaging a cell that synthesizes Type 1 collagen. The cell includes a first gene encoding collagen a1(I) tagged with a first fluorescent tag and a second gene encoding collagen a2(I) tagged with a second fluorescent tag.

### CROSS-REFERENCE TO RELATED APPLICATION

This claims priority to U.S. provisional Application No. 62/441,674, filed Jan. 3, 2017, which is incorporated by reference in its entirety.

### FIELD

This relates to the field of imaging and, more particularly, to using imaging techniques to characterize Type 1 collagen synthesis

### SEQUENCE LISTING

The application contains a Sequence Listing electronically submitted via EFS-web to the United States Patent and Trademark Office as a text file named “Sequence_Listing.txt.” The electronically filed Sequence Listing serves as both the paper copy required by 37 C.F.R. § 1.821(c) and the computer readable file required by 37 C.F.R. § 1.821(c). The information contained in the Sequence Listing is incorporated by reference herein in its entirety.

### BACKGROUND

There are twenty-eight different types of collagen. Type 1 collagen is the most abundant protein in the body. It is primarily responsible for giving bones, connective tissues, and organs their structure. Collagen has several different forms, which are identified by “Types.” Type 1 and Type 2 collagen have a fiber-like structure. It is found in the skin, tendons, ligaments, bones, retina, internal organs, and vascular system.

If the body produces excessive amounts of Type 1 collagen this can lead to a condition called fibrosis. Fibrosis is the presence of excess connective tissue in a region of the body. Excess connective tissue in an organ can interfere with the organ's functions. There are many diseases related to fibrosis, some of which include pulmonary fibrosis, cardiac fibrosis, renal fibrosis, arterial hardening, hepatic fibrosis with cirrhosis, scleroderma, and keloids and hypertrophic scars. Fibrosis-related diseases are a medical problem around the world.

### BRIEF SUMMARY

The manner in which the body synthesizes Type 1 collagen is known, but there are few, if any, techniques available to determine how or whether a drug candidate is effective at interfering with Type 1 collagen synthesis. This problem is overcome by the compositions and methods discussed here.

A first method includes fluorescence imaging a cell that synthesizes Type 1 collagen. Type 1 collagen is composed of two polypeptides; a1(I) and a2(I). The cell includes a first gene encoding collagen a1(I) tagged with a first fluorescent tag and a second gene encoding collagen a2(I) tagged with a second fluorescent tag.

This method may further include inserting the first gene and second gene into the cell.

In this method, the fluorescence imaging may be conducted while the cell is synthesizing Type 1 collagen.

A second method includes imaging Type 1 collagen synthesis. This method includes inserting a collagen a1(I) encoding gene into a cell, the collagen a1(I) encoding gene encoding a first fluorescent tag onto a collagen a1(I) polypeptide encoded by the collagen a1(I) encoding gene. It also includes inserting a collagen a2(I) encoding gene into a cell, the collagen a2(I) encoding gene encoding a second fluorescent tag onto a collagen a2(I) polypeptide encoded by the collagen a2(I) encoding gene. While Type 1 collagen synthesis occurs in the cell, the fluorescence of the first and second fluorescent tags is imaged.

Additional elements of these methods may include one or more of the following features.

The first fluorescent tag may fluoresce with a different color than the second fluorescent tag fluoresces.

The fluorescence imaging may be conducted while the cell is synthesizing Type 1 collagen.

The method may further include contacting the cell with a compound and determining whether the compound affects the Type 1 collagen synthesis.

The first fluorescent tag and second fluorescent tag preferably do not interfere with natural assembly of Type 1 procollagen.

An example of a drug screening process includes screening a compound for effectiveness against Type 1 collagen synthesis using a cell including a collagen a1(I) polypeptide having a first fluorescent tag thereon and a collagen a2(I) polypeptide having a second fluorescent tag thereon by contacting the cell with the compound and fluorescence imaging the cell.

In this process, the first fluorescent tag may fluoresce with a different color than the second fluorescent tag fluoresces.

In this process, the fluorescence imaging may be conducted while the cell is synthesizing Type 1 collagen.

This process may further include contacting the cell with a compound and determining whether the compound affects the Type 1 collagen synthesis.

In this process, the first fluorescent tag and second fluorescent tag preferably do not interfere with natural assembly of Type 1 procollagen.

In this process, the cell may include a first gene encoding the collagen a1(I) polypeptide having a first fluorescent tag thereon and a second gene encoding the collagen a2(I) polypeptide having a second fluorescent tag thereon.

The drug screening process may include fluorescence imaging the cell while the cell synthesizes Type 1 collagen prior to contacting the cell with the compound.

A composition component includes an artificially modified cell having a first gene that encodes collagen a1(I) tagged with a first fluorescent tag and a second gene that encodes collagen a2(I) tagged with a second fluorescent tag.

In the composition, the first fluorescent tag may fluoresce with a different color than the second fluorescent tag fluoresces.

In the composition, the first fluorescent tag and second fluorescent tag preferably do not interfere with natural assembly of Type 1 procollagen.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates four example constructs used in making up an example of the drug screening process and fluorescence imaging methods.

FIG. 2A is a confocal microscope image of COLa1(I)/mTagBFP2+WT COLa2(I)/mEmerald in a fixed cell taken by spin disk microscope.

FIG. 2B is a more detailed view of the squared region in FIG. 1A.

FIG. 3 is a series of confocal microscope images of fixed cells taken by a Zeiss confocal microscope expressing the following combinations: COLa1(I)/nnTagBFP2+WTCOLa2(I)/mEmerald (left panel); COLa1(I)/mTagBFP2+MUT COLa2(I)/mEmerald (middle panel) and COLa1(I)/mTagBFP2+COLa2(I)/mEmeraldΔC (right panel). The lower images are a more detailed view of the squared regions in the images directly above.

FIG. 4 is a series of confocal microscope images of fixed cells expressing the COLa1(I)/mTagBFP2+WT COLa2(I)/mEmerald tag. An untreated cell is shown in the top panel, while in the cell in bottom panel was treated for 24 hours with an antifibrotic drug.

FIG. 5 is a series of confocal microscope images illustrating high throughput screening of antifibrotic drugs by imaging of the fluorescent tags in live cells before and after addition of the drug.

### DETAILED DESCRIPTION OF EXAMPLE EMBODIMENTS

Cellular synthesis of collagen involves two important polypeptides: collagen a1(I) and collagen a2(I). Collagen a1(I) is encoded by a collagen a1(I) encoding gene called COL1A1. SEQ ID NO: 1 is the sequence of wild-type collagen a1(I) or COLa1(I).

Collagen a2(I) is encoded by a collagen a2(I) encoding gene called COL1A2. SEQ ID NO: 2 is the sequence if wild-type collagen a2(I) or COLa2(I).

The synthesized collagen a1(I) and collagen a2(I) combine into a protein called procollagen. In the extracellular matrix between cells procollagen is enzymatically converted into Type 1 collagen.

The cytogenic location of the COL1A1 gene is 17q21.33, which is the long (q) arm of chromosome 17 at position 21.33. Its molecular location is at base pairs 50,184,096 to 50,201,648 on chromosome 17 (Homo sapiens). See www.ncbi.nlm.nih.gov/gene/1277.

SEQ ID NO: 3 is the sequence of COL1A1.

The cytogenic location of the COL1A2 gene is 7q21.3, which is the long (q) arm of chromosome 7 at position 21.3. Its molecular location is at base pairs 94,394,561 to 94,431,232 on chromosome 7 (Homo sapiens). See www.ncbi.nlm.nih.gov/gene/1278.

SEQ ID NO: 4 is the sequence of COL1A2.

A molecular system which can visualize biosynthesis of Type 1 collagen in live cells and reproduce aspects of the process has been developed. The system is based on insertion of a first fluorescence protein gene tag into a human procollagen a1(I) cDNA and second fluorescence protein gene tag into human procollagen a2(I) cDNA. When these coding sequences are expressed, the full size collagen propeptides are fluorescent-labeled and they fold into Type 1 procollagen protein.

Fluorescence occurs when a substance absorbs electromagnetic radiation and emits light in response. Fluorescence can be visualized with conventional imaging techniques, including fluorescence microscopy, confocal microscopy, and high content plate image readers, among others.

A fluorescent tag, also called a fluorescent label or fluorescent marker among other names, is a molecule that can be attached, via covalent bonding or otherwise, to a molecule intending to be imaged. The tag includes at least one functional group that will fluoresce when excited by electromagnetic radiation.

By attaching a fluorescent tag to a target molecule, the location of the target molecule becomes visible when the tag fluoresces. This allows the fluorescent tag to act as a visible marker for the target molecule.

There are many different fluorescent tags that may be used to practice the methods and compositions described here. Accordingly, rather than providing an exhaustive list of possible tags, a few possible tags are now described in more detail.

The fluorescent tag may be, for example, a fluorescent protein or “FP”. A “BFP” is a blue fluorescent protein. A “GFP” is a green fluorescent protein. An “RFP” is a red fluorescent protein. There are also many examples of fluorescent proteins of different colors including yellow, orange, and cyan.

A conventional BFP used to image cellular components is called mTagBFP. Subach, et al., Chem. Biol. Vol. 17, 333-42 (2010).

A variant of mTagBFP is mTagBFP2. Subach et al., PloS ONE, Vol. 6, Iss. 12 e28674 (2011). This fluorescent protein has an excitation band at about 400 nm and an emission band at about 454 nm.

Another example of a BFP is EBFP2, which has an excitation band at about 383 nm and an emission band at about 448 nm.

An example a useful GFP is mEmerald, which has an excitation band at about 487 nm and emission band at about 509 nm, quantum yield of 0.68 and is a monomer with 1.16-fold higher brightness than EGFP (mutations S65T, S72A, N149K, M153T, I167T).

Many cloning vectors expressing fluorescent proteins are commercially available. Cloning vectors for mEmerald are available from ThermoFisher Scientific.

There are a plethora of fluorescent proteins that may possibly be used and which commercially available and/or reported in the literature, thus, this disclosure is not intended to list all possible examples. Those skilled in the art may select fluorescent proteins for use based on their preferences and objectives. Shaner et al., in Journal of Cell Science, 120, 4247-4260 (2007), provide a review of many different fluorescent proteins. Shaner et al., is incorporated by reference in its entirety.

A composition component of this disclosure includes an isolated gene that encodes for a wild type human collagen a1(I) polypeptide (SEQ ID NO: 1) having a fluorescent tag in the peptide sequence thereof.

Another composition component of this disclosure includes isolated gene that encodes for a wild type human collagen a2(I) polypeptide (SEQ ID NO: 2) having a fluorescent tag in the peptide sequence thereof.

Another composition component of this disclosure includes an isolated polypeptide comprising wild type human collagen a1(I) (SEQ ID NO: 1) having with a fluorescent tag in the sequence thereof.

Another composition component of this disclosure includes an isolated polypeptide comprising wild type human collagen a2(I) (SEQ ID NO: 2) having a fluorescent tag in the sequence thereof.

Another composition component of this disclosure includes an artificially modified cell having a first gene that encodes for collagen a1(I) (SEQ ID NO: 1) tagged with a first fluorescent tag and a second gene that encodes for collagen a2(I) (SEQ ID NO: 2) tagged with a second fluorescent tag.

The collagen a1(I) polypeptide having a first fluorescent tag thereon and the collagen a2(I) polypeptide having a second fluorescent tag thereon may be prepared by having a cell that expresses the respective polypeptide from modified genes that encode for collagen a1(I) collagen a2(I) polypeptides with the desired fluorescent tag thereon. This can be accomplished, for example, using cDNA that encodes for the tagged polypeptide. Accordingly, the cDNA includes a fluorescent tag encoding section in its sequence as well as the sequence encoding for the desired polypeptide.

The tag may be selected from tags that do not substantially interfere with the natural assembly of Type 1 procollagen within the cell. This allows the Type 1 procollagen to assemble normally as if untagged. By allowing the Type 1 procollagen to assemble normally, collagen synthesis will occur as it normally does and the imaging results will accurately provide information on natural collagen synthesis.

The placement of the tag within the polypeptides may be such that they do not substantially interfere with normal assembly of Type 1 procollagen.

One or more of these composition components may be used in various useful method which are now described.

An example of such a method includes fluorescence imaging a cell that synthesizes Type 1 collagen. The cell includes the first gene encoding collagen a1(I) tagged with a first fluorescent tag and the second gene encoding collagen a2(I) tagged with a second fluorescent tag.

In another example of such a method the collagen a1(I) encoding gene is inserted into a cell and the collagen a2(I) encoding gene is inserted into the cell. The collagen a1(I) encoding gene encodes the first fluorescent tag onto a collagen a1(I) polypeptide encoded by the collagen a1(I) encoding gene. The collagen a2(I) encoding gene encodes the second fluorescent tag onto a collagen a2(I) polypeptide encoded by the collagen a2(I) encoding gene. The cell is fluorescence imaged while Type 1 collagen synthesis occurs in the cell.

These genes may be inserted into the cell via a conventional genetic modification mechanism such as, for example, by using a viral vector.

In these methods, the synthesis of Type 1 procollagen may be visualized by measuring or imaging overlapping fluorescence signals from the first and second fluorescence tags. This allows determination of qualitative and quantitative aspects of Type 1 collagen synthesis in real time.

These methods may be used in research on the mechanism of Type 1 collagen synthesis and for screening of compounds as antifibrotic drugs, for example.

The methods allow for visualization of subcellular localization, trafficking and folding of two collagen propeptides (comprising Type 1 procollagen) in living cells. The conventional techniques of immunostaining and cell fractionation do not provide such detail. The ability of the imaging system to allow for visualization of collagen synthesis makes it an useful tool for discovery of chemical compounds that can interfere with Type 1 collagen synthesis.

Drug screening is a technique in which a cell or group of cells is contacted with a drug, then a parameter of the cell such apoptosis or production of a particular protein is monitored to determine the effect of the drug on the cell. This type of drug screening procedure is sometimes called phenotypic drug screening.

An example of a drug screening process includes screening a compound for effectiveness against Type 1 collagen synthesis using a cell including a collagen a1(I) polypeptide having a first fluorescent tag thereon and a collagen a2(I) polypeptide having a second fluorescent tag thereon by contacting the cell with the compound and fluorescence imaging the cell.

The term contacting refers to bringing the cell(s) into direct physical contact with the compound. Contacting may be achieved in many different ways such, for example, as by mixing the drug with the cell(s) being contacted by a conventional mechanism.

This method is useful for determining how or whether the compound interferes with Type 1 collagen synthesis. The tagged cell(s) may be imaged before being contacted with the compound to visualize collagen synthesis without the compound. The tagged cell(s) may also be imaged during and/or after being contacted with the compound to visualize collagen synthesis after treatment with the compound. The difference indicates how the compound affects collagen synthesis.

This screening method will allow for screening of compounds as therapeutic agents. This may be accomplished on a small scale by testing a few compounds or on a large scale by testing a library of compounds.

If desired the methods and drug screening process may be automated by incorporating a computer program that executes the steps.

The methods may also utilize additional constructs which are not subjected to the regulation and serve as controls for specificity. The constructs may be made adenoviral vectors, allowing visualization and quantification of collagen biosynthesis in spatial and temporal manner in wide variety of living cells.

### EXAMPLE

This section provides an example that illustrates various technical details of example embodiments of the compositions and methods. The scope of possible embodiments is not limited to what this example teaches.

To develop the cells for fluorescence imaging, chimeric genes were constructed including an mTagBFP2 sequence incorporated within the sequence of human collagen a1(I) polypeptide (COLa1(I)/nnTagBFP2, blue fluorescence) and mEmerald sequence incorporated within the human collagen a2(I) polypeptide (COLa2(I)/mEmerald, green fluorescence). The chimeras were constructed in such a way that the sequences of the fluorescent proteins do not substantially interfere with normal synthesis, modifications and assembly of the collagen polypeptides.

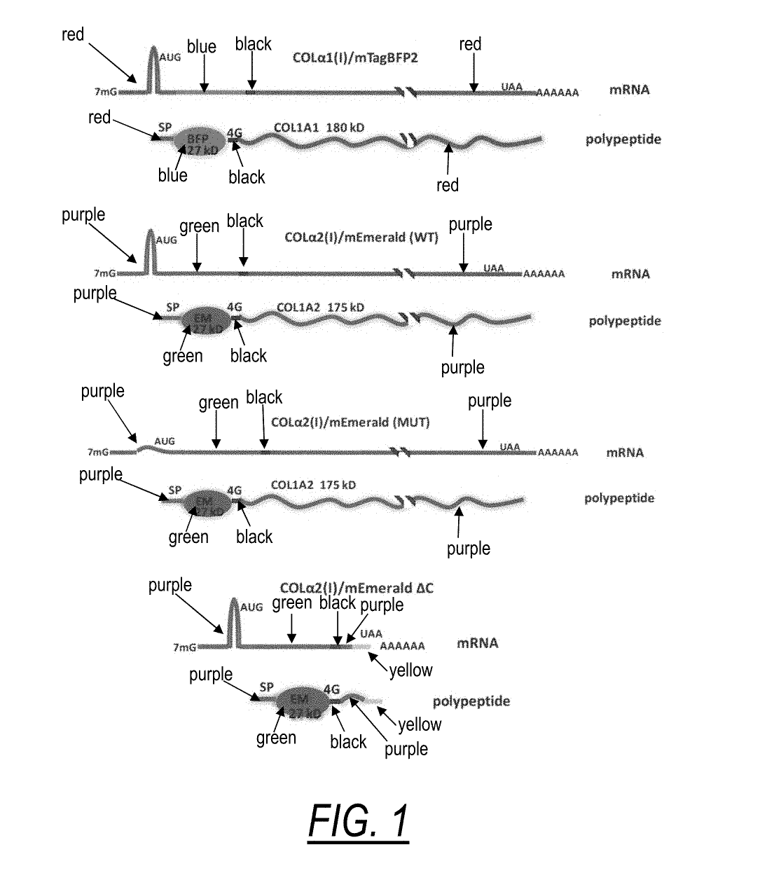

FIG. 1 illustrates four example constructs making up an example of the imaging system. A schematic representation of mRNA and polypeptides encoded by these mRNAs is shown. Red=sequences of collagen a1(I). Blue=mTagBFP2. Black=4G, the 4 glycine linker between fluorescent tag and collagen sequences. Purple=sequences of collagen a2(I). Green=mEmerald. Yellow=nonsense sequence in the truncated a2(I)/Emerald. SP stands for signal peptide. AUG and UAA stand for translation start and stop codons, respectively. 7mG=mRNA cap. AAAAA=poly-A tail.

The visualization of the individual collagen polypeptides was achieved by fluorescence imaging in blue and green channels and their co-localization by double fluorescence imaging.

The specificity of the system was controlled by utilization of two control constructs: MUT COLa2(I)/mEmerald and COLa2(I)(AC)/mEmerald. The 5′ stem-loop is the sequence element of collagen mRNAs which regulates translation of a1(I) and a2(I) polypeptides, therefore, the control construct MUT COLa2(I)/mEmerald will express a2(I)/mEmerald chimera from the mRNA lacking this element. This construct, in combination with COLa1(1)/mTagBFP2, visualizes synthesis without proper regulation.

The other control construct expresses a truncated a2(I)/mEmerald chimera. The short a2 polypeptide produced by this construct cannot assemble into the collagen triple helix and this construct visualizes the fluorescence in the absence of active Type 1 collagen production as a negative control.

The constructs were designed as adenoviruses.

Construction of the COLA1(I)/mTagBFP2 Gene

Step 1.

The COLA1(I)/mTagBFP2 gene was prepared from a starting construct of X07884 (ATCC 95498, 95499 in pUC18 vector).

Step 2.

A 4.5 kb EcoRI-EcoRI fragment from step 1 was recloned into the EcoRI site of a pCDNA3 vector in 3′ to 5′ orientation.

Step 3.

By site directed mutagenesis an AgeI site was created at codon 26 using the following primers:

(SEQ ID NO: 5)
GGCCAAGAGGAAGGACCGGTCGAGGGCCAAGA
and
(SEQ ID NO: 6)
TCTTGGCCCTCGACCGGTCCTTCCTCTTGGCC

Step 4.

The cDNA for mTagBFP2 protein was PCR amplified with primers

(SEQ ID NO: 7)
GCCACCGGTAGTGTCTAAGGGCGAAGAGCT
and
(SEQ ID NO: 8)
GCCACCGGTGCACCTCCGCCCCCATTAAGCTTGTGCCCCAGTT,

cut with AgeI, and ligated into AgeI site of clone from the previous paragraph. Orientation and frame was verified by sequencing.

Full size human collagen a1(I) cDNA was reconstructed by cloning of ClaI-HindIII fragment from step 1 into the clone obtained in step 4.

Step 5.

Full size of human collagen a1(I) cDNA was reconstructed by cloning of ClaI-HindIII fragment from step 1 into clone obtained in step 4.

For making an adenovirus expressing COLa1(I)/mTagBFP2, pAdTrack-CMV vector was modified by removing the HpaI fragment (containing the GFP cassette) and religating the vector. The NotI-SalI fragment of the clone in step 5 was recloned into KpnI and XhoI of the modified pAdTrack-CMV vector. The adenovirus was assembled by recombination of this clone with pAdEasy in BJ5 183 E. coli cells and packaged in HEK293 cells and amplified using the standard procedures.

Construction of the WT COLA2(I)/mEmerald and MUT COLA2(I)/mEmerald Genes

Step 1.

The starting construct was BC042586 (cDNA clone MGC:30044 IMAGE:4803351 in vector pSPORT6.ccdb), purchased from GE Healthcare-Dharmacon.

Step 2.

The cDNA for collagen a2(I) in this construct was shortened by cutting with XcmI and HindIII and removal of fragments and re-ligation after end blunting.

Step 3.

The two double stranded oligonucleotides, one for making WT COLa2(I)/mEmerald and one for MUT COLa2(I)/mEmerald were purchased; the sequence of these oligonucleotides is below:

WT COLA2(I)/mEmerald oligo seq:

(SEQ ID NO: 9)
5′ TCAGCTTTGTGGATACGCGGACTTTGTTGCTGCTTGCAGTAAC
CTTATGCCTAGCAACATGCCAATCTTTACAAGAGCGAAACACCTAT
GCGCCTGAAACAACGACGAACGTCATTGGAATACGGATCGTTGTAC
GGTTAGAAATGTTCTC 3′
(SEQ ID NO: 10)
3′ GATCCTGTAAGAAAGGGCCCTAGGACATTCTTTC 5′

MUT COLA2(I)/mEmerald oligo seq:

(SEQ ID NO: 11)
5′ TCAGCTTTGTGGATACGCGGACTTTGTTGCTGCTTGCAGTAAC
CTTATGCCTAGCAACATGCCAATCTTTACAAGAGCGAAACACCTAT
GCGCCTGAAACAACGACGAACGTCATTGGAATACGGATCGTTGTAC
GGTTAGAAATGTTCTC 3′
(SEQ ID NO: 12)
3′ GATCCTGTAAGAAAGGGCCCTAGGACATTCTTTC 5′

These oligonucleotides were cloned into BpII and ApaI sites of the truncated collagen a2(I) cDNA from step 2.

Step 4.

The cDNA for Emerald protein was amplified by PCR with primers:

(SEQ ID NO: 13)
CGCAGATCTAGTGAGCAAGGGCGAGGAGCT
and
(SEQ ID NO: 14)
CGCAGATCTCCACCTCCGCCCTTGTACAGCTCGTCCATGCC

cut with BglII and ligated into BamHI site of WT and MUT constructs from step 3. Orientation and frame was verified by sequencing. The WT construct from this step is used as COLA2(I)(AC)/mEmerald.

Step 5.

The full size WT COLA2(I)/mEmerald and MUT COLA2(I)/mEmerald were reconstructed by cloning of KpnI and PmlI fragment from clones in step 4 back into the original vector from step 1.

For making adenoviruses expressing WT COLA2(I)/mEmerald and MUT COLA2(I)/mEmerald, the modified pAdTrack-CMV vector described above was used. The KpnI-NotI fragment of clones in step 5 was recloned into KpnI and NotI of the modified pAdTrack-CMV vector. For COLA2(I)/mEmeraldΔC, the same procedure was followed but using the clone from step 4. Adenoviruses were assembled as described above.

Preparation of Cells

Three combinations of adenoviruses were used:
COLα1(I)/mTagBFP2 and WT COLα2(I)/mEmerald: images normal, regulated Type 1 collagen synthesis;
COLα1(I)/mTagBFP2 and MUT COLα2(I)/mEmerald: images collagen synthesis without regulation; and
COLα1(I)/mTagBFP2 and COLα2(I)/mEmeraldΔC: images absence of productive synthesis.

Adenoviruses were mixed in a ratio determined by the viral titer and added to human lung fibroblasts in culture. After 48 hours the cells were either fixed in 3.7% formalin, mounted on glass slides and imaged or they were directly imaged as live cells.

Imaging of Cells

Confocal imaging of cells was performed either by Zeiss LSM 880 confocal microscope or Nikon Eclipse Ti laser spin disk confocal microscope in blue and green channels.

The drug used in the confocal microscopy imaging experiments was compound 60D17 of U.S. Pat. No. 8,729,090, which is incorporated by reference. Compound 60D17 has the following formula.

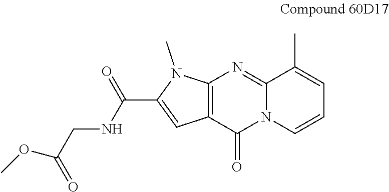 [SMILES: COC(=O)CNC(=O)c1cc2c(=O)n3cccc(C)c3nc2n1C]

Experimental results are now discussed with reference to the figures.

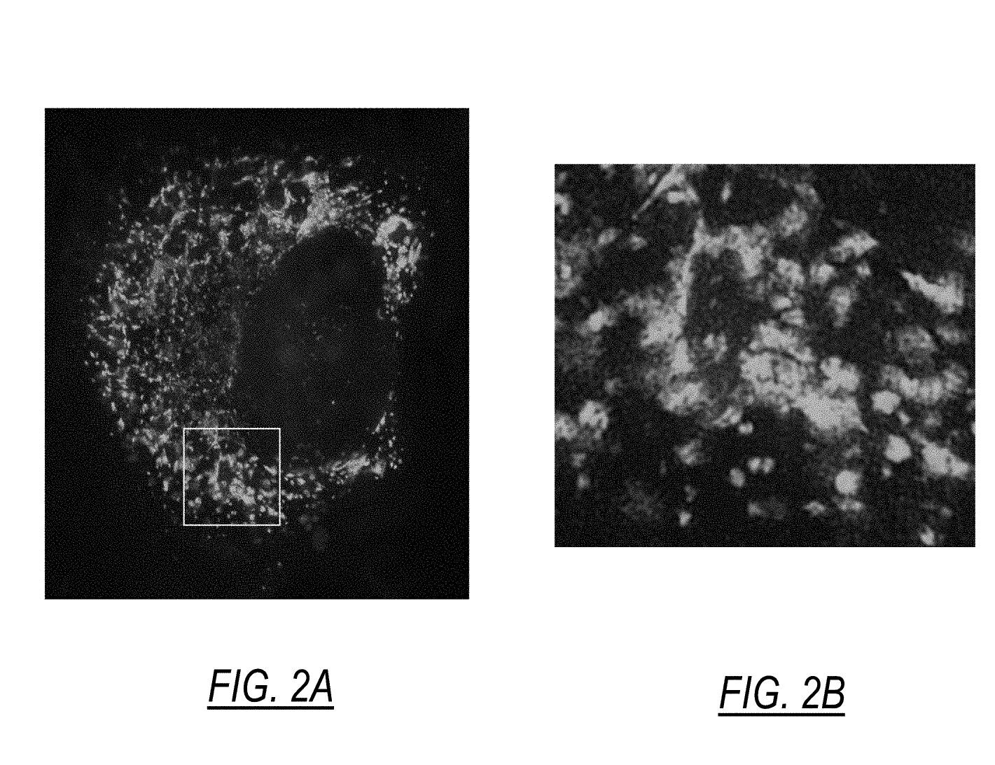

FIGS. 2A and 2B are confocal microscope images of COLa1(I)/mTagBFP2+WT COLa2(I)/mEmerald in a fixed cell taken by spin disk microscope. As can be seen, the fluorescent tags allow for visualization of regulated Type 1 collagen synthesis which is clustered into discrete vesicles.

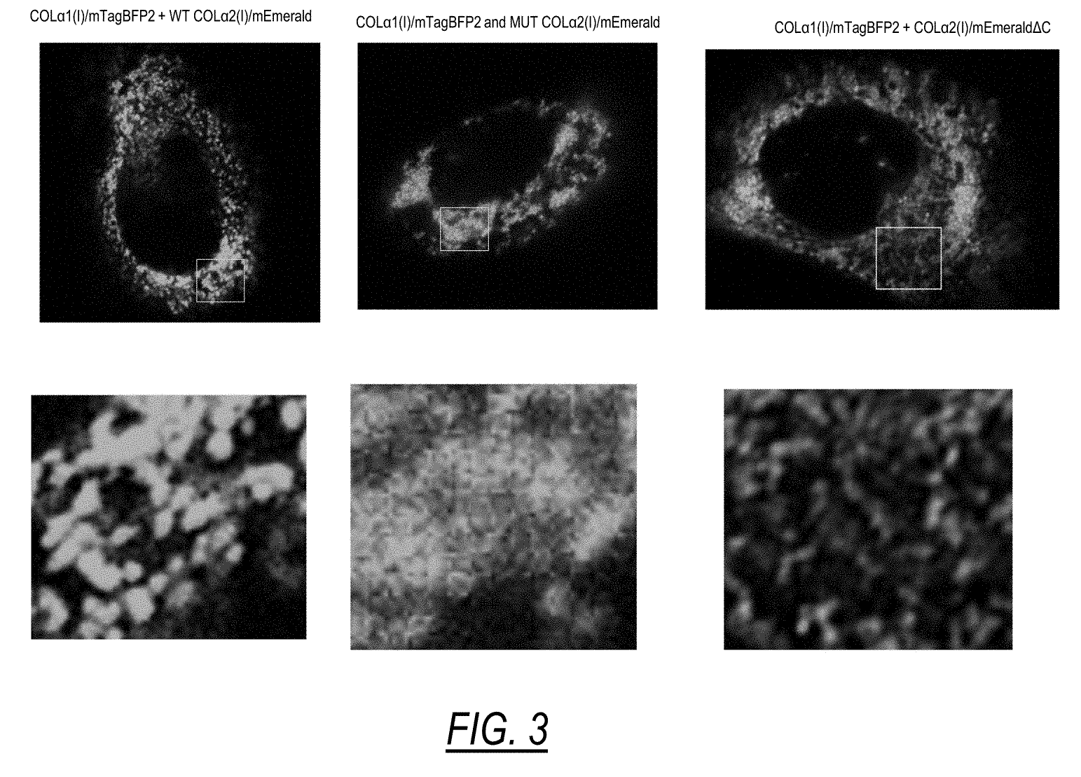

FIG. 3 is a series of confocal microscope images of fixed cells taken by a Zeiss confocal microscope expressing the following combinations: COLa1(I)/mTagBFP2+WTCOLa2(I)/mEmerald (left panel); COLa1(I)/mTagBFP2+MUT COLa2(I)/mEmerald (middle panel) and COLa1(I)/mTagBFP2+COLa2(I)/mEmeraldΔC (right panel). The lower images are a more detailed view of the squared regions in the images directly above.

The regulated, clustered collagen biosynthesis is only visible with COLa1(I)/mTagBFP2+WT COLa2(I)/mEmerald combination, while the COLa1(I)/mTagBFP2+MUT COLa2(I)/mEmerald shows lack of vesicular organization. With COLα1(I)/mTagBFP2+COLα2(I)/mEmeraldΔC there is uncoupling of blue and green fluorescence.

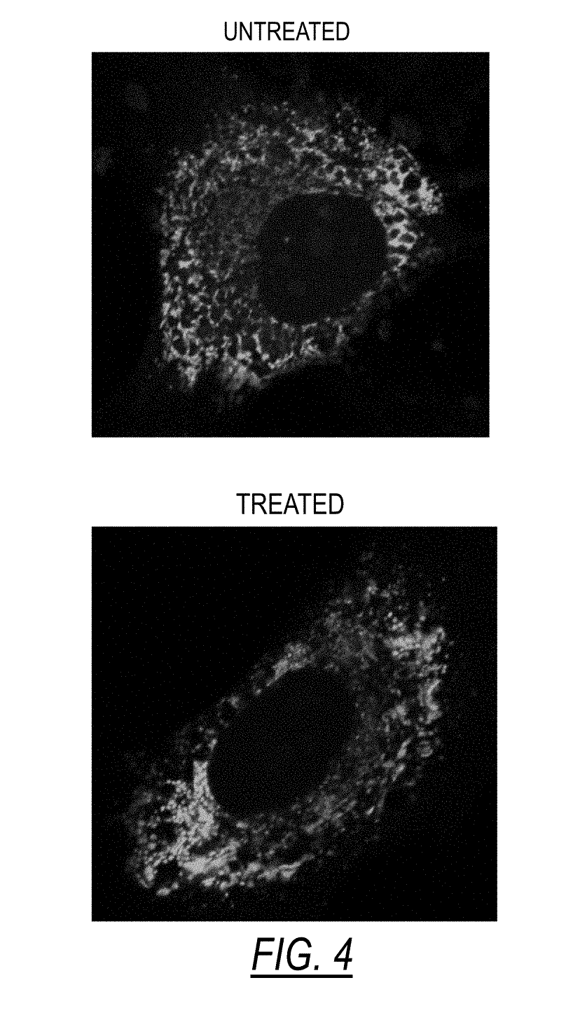

FIG. 4 is a series of confocal microscope images of fixed cells expressing the COLa1(I)/mTagBFP2+WT COLa2(I)/mEmerald tag. An untreated cell is shown in the top panel, while in the cell in bottom panel was treated for 24 hours with an antifibrotic drug. After the drug treatment, the images are clear that Type 1 collagen biosynthesis has been perturbed.

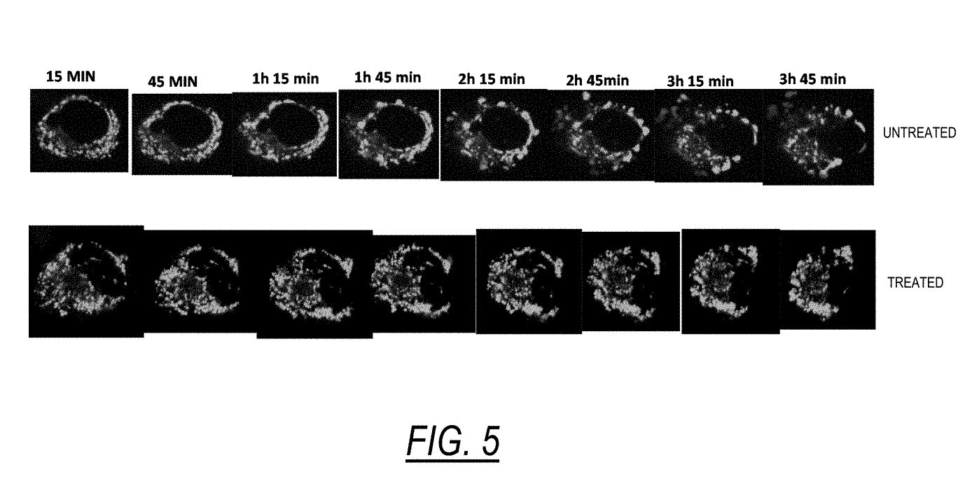

FIG. 5 is a series of confocal microscope images illustrating high throughput screening of antifibrotic drugs by imaging of the fluorescent tags in live cells before and after addition of the drug. The spin disk images show a live cell expressing the COLa1(I)/mTagBFP2+WT COLa2(I)/mEmerald tag at the time points indicated without treatment (upper panels) and with treatment (lower panels). The ability of the fluorescence tags to allow for detecting the changes in collagen biosynthesis confirm that the system may be employed to screen for antifibrotic drugs.

This disclosure describes exemplary embodiments, but not all possible embodiments of the methods and compositions. Where a particular feature is disclosed in the context of a particular method or composition, that feature can also be used, to the extent possible, in combination with and/or in the context of other embodiments. The compositions and methods may be embodied in many different forms and should not be construed as limited to only the embodiments described here.

That which is claimed is:

1. A method of imaging Type 1 collagen synthesis, the method comprising:
inserting a collagen a1(I) encoding gene into a cell, the collagen a1(I) encoding gene encoding a first fluorescent tag onto a collagen a1(I) polypeptide encoded by the collagen a1(I) encoding gene;
inserting a collagen a2(I) encoding gene into a cell, the collagen a2(I) encoding gene encoding a second fluorescent tag onto a collagen a2(I) polypeptide encoded by the collagen a2(I) encoding gene; and
imaging the fluorescence of the first and second fluorescent tags while Type 1 collagen synthesis occurs in the cell.

2. The method of claim 1, further comprising contacting the cell with a compound that interferes with Type 1 collagen synthesis and imaging the fluorescence of the first and second fluorescent tags while Type 1 collagen synthesis occurs in the cell for determining how the compound interferes with Type 1 collagen synthesis.

3. The method of claim 1, wherein the first fluorescent tag fluoresces with a different color than the second fluorescent tag.

4. The method of claim 1, wherein the first fluorescent tag and second fluorescent tag do not interfere with natural assembly of Type 1 procollagen.

5. A method comprising fluorescence imaging a cell that synthesizes Type 1 collagen, the cell including a first gene encoding collagen a1(I) tagged with a first fluorescent tag and a second gene encoding collagen a2(I) tagged with a second fluorescent tag.

6. The method of claim 5, wherein the first fluorescent tag fluoresces with a different color than the second fluorescent tag fluoresces.

7. The method of claim 5, wherein the fluorescence imaging is conducted while the cell is synthesizing Type 1 collagen.

8. The method of claim 5, further comprising contacting the cell with a compound and determining whether the compound affects the Type 1 collagen synthesis.

9. The method of claim 5, wherein the first fluorescent tag and second fluorescent tag do not interfere with natural assembly of Type 1 procollagen.

10. The method of claim 5, further comprising inserting the first gene and second gene into the cell.

11. A drug screening process comprising screening a compound for effectiveness against Type 1 collagen synthesis using a cell including a collagen a1(I) polypeptide having a first fluorescent tag thereon and a collagen a2(I) polypeptide having a second fluorescent tag thereon by contacting the cell with the compound and fluorescence imaging the cell.

12. The drug screening process of claim 11, wherein the first fluorescent tag fluoresces with a different color than the second fluorescent tag fluoresces.

13. The drug screening process of claim 11, wherein the fluorescence imaging is conducted while the cell is synthesizing Type 1 collagen.

14. The drug screening process of claim 11, further comprising contacting the cell with a compound and determining whether the compound affects the Type 1 collagen synthesis.

15. The drug screening process of claim 11, wherein the first fluorescent tag and second fluorescent tag do not interfere with natural assembly of Type 1 procollagen.

16. The drug screening process of claim 11, wherein the cell includes a first gene encoding the collagen a1(I) polypeptide having a first fluorescent tag thereon and a second gene encoding the collagen a2(I) polypeptide having a second fluorescent tag thereon.

17. The drug screening process of claim 11, further comprising fluorescence imaging the cell while the cell synthesizes Type 1 collagen prior to contacting the cell with the compound.

18. A composition comprising an artificially modified cell having a first gene that encodes collagen a1(I) tagged with a first fluorescent tag and a second gene that encodes collagen a2(I) tagged with a second fluorescent tag.

19. The composition of claim 18, wherein the first fluorescent tag fluoresces with a different color than the second fluorescent tag fluoresces.

20. The composition of claim 18, wherein the first fluorescent tag and second fluorescent tag do not interfere with natural assembly of Type 1 procollagen.


SEQUENCE LISTING (14 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens
MLSFVDTRTLLLLAVTLCLATCQSLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPAGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPPGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAPGAVGAPGPAGATGDRGEAGAAGPAGPAGPRGSPGERGEVGPAGPNGFAGPAGAAGQPGAKGERGAKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGLKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMDAIKVYCDFSTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADQEFFVDIGPVCFK

SEQ ID NO: 3 - DNA, 18360 bp, Homo sapiens
aagcttaaaaaggagtaggcgggaggcgggggcacgatggttccccagaccaccccagcccaggggggttgtgttctggggattcaggaggtcgaggcacccagtccgtggtgagcagggccaggtctccttccttcccaagagcacggtgcgagtgccttccccacagctctcccaggtgtctgtctccctcactcccccagggccaggacagcgagcagggagcacgccttcctcctggtccctcccagaccctcgccaggctcagcagacccctctcctgttgcgttgctccttcggcaggaggtcagcgtgggtgagaagggagcagaggccagatgacccttcatgtcagttcaaggagaggcccccaggtcctcctcacgggttccactcccctcaccctcccagtatccatgagcatcccaccgccaccaagtgcagggcatccagggggtggagagtggatgggcttgaaggggagtgggtatgaggtttggacaaccccagatgtgaagagtgtgtgtgtgtgttgtgcacgtggattgcctgtatggtattgcaggggagaagaggatttggaaaaggtgcaaggaaattgcaagaatagaaaatgaaagaggaagtagaaaggtttgcggtataagaagggtgagagaaaggggagtggggttatggagggatctagaagagaatagtggggagggaaagggcaggaatggaaggcaagcccatcaccccaaagccccttccccatgtctacccctcccccaactccaaagaaggaaattaggaggccactgcggattatgtttgggtcatttccacatgctttattccagcaatcaaaataattaaaaacatctcaaattattatacacatacaaaataggtacagagtcttttgcttcctcccacccctagggggaaaaactgctttgtgctttgggaagttgtctctgaaacccggggacagaggacgcaggacagactaggagggagccgggaggatgggctgcagctgtggaggagggtttcagaggagagaggtcggagagcagaggcctgagaagccagaggcaggtggagagagggtggaaagtgagcagcgggctgggctggagccgcacacgctctcctcccatgttaaatagcacctttagaaaaattcacaagtccccatccacaaaaaaaaaaaaaaaaaaagaaaaatatcaaggaataaaaatagactttgaacaaaaaggaacatttgctggcctgggggggcatctcaatttctatagcaccagtgattccctccccaccccacccatcacatagatgtagcacctttggtataaaatggggagccgcttccaccctgcccccatccccgcccccaggcagttgccccggtgacacatcaagacaagaacgaggtagtctttcagcaacacagttacacaaggaacagaacagtctctcccgcccagccctgcggcacaagggattgacacgcgttccccaaatccgatgtttctgctttgtcgtggcccttcctgactctcctccgaacccagtgaggggctggtggctcccccggcatgaccccctcaaaaacgaaggggagatgttgcaagagccatgggagcgccagatggcaaggcttctttggcagtctgagaaccccaggtcccccagggcctgggggtgctgggcgggcaggagcgggctgagggtgggggccacttgggtgtttgagcattgcctttgattgctgggcagacaatacattgtttcctgtgtcttctggggagacagatttgggaaggagtggaggggaggccccaaggggggtgtggagaaaggagcagaaagggcagcattggggtttcataagcccaacgggcagaaagggacttacccccgcatgggtcttcaagcaagtggaccaagcttccttttttaaaaagttatttatttattctttttttttttttttttttggtaaggttgaatgcacttttggtttttggtcatgttcggttggtcaaagataaaaactaagtttgagagatgaatgcaaaggaaaaaaatattttccaaagtccatgtgaaattgtctcccattttttggcttttgagggggttcagtttgggttgcttgtctgtttccgggttggggggaaagttggttgggtgggagggagccaggttgggatggagggagtttacaggaagcagacagggccaacgtcgaagccgaattcctggtctggggcaccaacgtccaagggggccacatcgatgatgggcaggcgggaggtcttggtggttttgtattcaatcactgtcttgccccaggctccggtgtgactctggggtggggcggagacaacgggagggggcattaccacgtgggagtgatggagagagggcactatggcctggccaaaaagcccaaggccggagaggtggggccctgcctggggattctgggcactcaccgtgcagccatcgacagtgacgctgtaggtgaagcggctgttgccctcggcgcggatctcgatctcgttggagccctggaggagcagggccttcttgaggttgccagtctgctggtccatgtaggccacgctgttcttgcagtggtaggtgatgttctgggaggcctcggtggacatcaggcgcaggaaggtcagctggatggccacatcggcagggtcggagccctggccgccatactcgaactgcaggggaggggagagagggaagagtgagccgctatgcgggaacctctagtcctgcctggcctccctgtccagggtcctcagagagctgcccaatgcaccgttatatcgagaggaggcaccacctgcccatcggcagcctgtgtctgaaccactatcagggacctgagacccctgcctcccagcccagctctgtccatcacccttagcagagacctactccaggacttcatgtcccttctgagcactgggctagcccatctcctaacactggctctgaggtccagctcacgcacctggaatccatcggtcatgctctcgccgaaccagacatgcctcttgtccttggggttcttgctgatgtaccagttcttctgggccacactgggctgagtggggtacacgcaggtctcaccagtctccatgttgcagaagactttgatggcatccaggttgcagccttggttggggtcaatccagtactctcctgtggtagggcagggcaagatggagtcagggaaagggagcagccagcaccatatggtaggggcacatatgggcatggggaccctggcatggcaggagtaggagggagggagaggctagggcaggccctcaccactcttccagtcagagtggcacatcttgaggtcacggcaggtgcgggcggggttcttgcggctgccctctgggctccggatgttctcgatctgctggctcaggctcttgagggtggtgtccacctcgaggtcacggtcacgaaccacattggcatcatcagcccggtagtagcggccaccatcgtgagccttctcttgaggtggctggggcaggaagctgaagtcgaaaccagcgctgggaggaccagggggaccaggaggtccaggagggccggggggaccctgcacagagagggaagagagtggggattaccggcatccaagtgctttgggggctggagggccatgagcagaggggatgaggggctacatacaacaggaccagcatcaccagtgcgaccgcgaggaccagggggcccaatggggccagggagaccgttgagtccatctttgccaggagcaccagcagagccagggggaccctggagtgggggaaatggtttgagaaaggctgccagaagcccgaacaaccccagctctggaggagaggcccaccaccctccctgctggctctggccccacggctcatagagccagcctcagcctctttccataggacatgccatagctcctcccagccgctgagccaggggaggacggattggggagcagagaggccaaagctagatcagattgtttgttcaggattcttcctctctttgcccactccctgtccctgaacccttctccagagaggcaaagggtgcctgggtccctggcaagggtccccgaggtgagcctgggcttggggctcaggaagaggagagagaaggcatgacttactcggggaccagcaggaccagaggctccagagggaccttgttcaccaggagagccctgaaggacagataaaaaaggcagttcaggcccagtgagcgtcaaatgtagcctgagggcctggctggagagacaagggcagtgtgggcaacccatgccccttcattattctggaaatctagcccgagggtgtccataggcagagacctctcccataatccctctctgtgtacccctcaccctctcagaagctacttggacatgcccacaaatcttcagaccccagtccccactagggaggggaaagaatgactatccagagggggaaactgaggcgaagctccccctcctatcccacagcacagcatggggactggggaggggctgagcatacttacaggagggccagggggaccctggaggccagagaagccacggtgaccctttatgcctctgtcgccctgttcgcctgtctcacccttgtcaccacgggggccttggggtccctagaagagagaaagggacaaactgtcaggcggaagttccattggcatcgagtggggcactgtctgcatctgtagagttctaaaggcatgggggacacagcagggtacttacggcggggccacgggcgccaacagggccgacaggaccggcgggaccagcaggaccctggggagagcaaggaaagcatgagctcttggccagggaaggctgaggctggggctgcaggatgaggcctcccctctgctggatctctctctcctcagctgcttcccactgtggccatctctcccaactcccagggaaacctccccactgcaatcttcacgggagctggggccaactcatgggagaggcggtcctgtctgggagaggggacttggggctgagctttaactcagttttttggattaaggccctgacatcttgcaggatctcctttcctccccctgcctgggtgaagtccgacacccatccccaggcctctaaggaggcctgaagagtccctggcctgaccaggtacagggaactggagcccagctacttacagtctcaccacgatcaccactcttgccagcagggccaacggggccaggggcaccaggagcaccaggagcaccagggggtccagcggggccggtctcaccacggtcaccctggcggggagagcaggggaatatgggtcagccccgggtgaagggccaggatggggcagggaagcagcagacaaggctgtggtcatggagtgttgccatcttaccttggcgccaggagaaccgtctcgtccaggggaaccttcggcaccaggagccccctgcagagagagagagagagaagtgagagtcagccggggaagagggcttaggcaaggccacaatggccatgctgagggtactggcatgggggctggggactgctcacctcacgtccagattcaccagggggtccagccaatccaggggggcccatgggaccagggggaccacgttcaccacttgctccagagggaccttgtttgccaggttcaccctaagggagaagaaagagtcaggccagagatagggtctgggaggacccttgagtccgctggagtcatctctaccaaatctgttctccttggctccgccccactccaagtcctgtgatggtttttctcagggccccccaaggtgaggggggcacttacagaggggccaggaagaccagggaagcctctctctcctctctgaccaggcaggccgaccacaccacgctgtccagcaataccttgaggcccgggagtaccaggagcaccctttgggaggcaaacaggggtgaggtgccagagagcagcacagggacccctccccagctctgcacacctccggagctgcagagatctgagctggcacttacagcaggaccatcagcaccaggggatcctttctcgccagcagggccagggggaccagggggaccaacttcaccaggacgtccagcagggccagtctcaccacggggacctttgccgccttctttgccagcaggaccaggagggccagggggtccagcatttccctggatgaggataggaggggctgtcagactccagggggctctggtgcatcattgggtcctcagtcagccccaccatccttctggcagcccccacccagcacccccaacctagagcagtggactctgctgcagagacttacagaggggccaggaggaccgactcggccagcagcaccagggaaaccagtagcaccctggaaggagagaacataggaacagtcagagggagaacagccaactcatccgacccagctgccctcacctgccaccgctgcctggggagaggggagaggctcaacagagaggcgggtgatactcacagggggaccagcgctgccgcgagcacctttggctccaggagcaccaacattaccctgtaggagagcacagaggcatcaagcctggacccgtcctgggtcccagcccaccagcctcgtgggcacagagggccaagccactcacaatggggccagggggtccagcgggtccggcagggccagggggaccagcatcgcctttagcaccagcatcaccaggttcgcctttagcaccaggttggccgtcagcaccctgggggaggaagcagggcggtgaatggagggaaggaggcaggagtttccactacctgggggaggagcagtaatggaggcaggaagatgcttgggtgggaaacaatcccgtctccacccttctcccctgaggatggctgacgcctttgtcctcattccgtccctcgaggtcccaggtcccagtcggtgatgaaaaatgatgggggtcttggtactcacaggggggccagcaaagccagcagggccggggggaccaggctcaccacggtctccctagaagaaaaggagtcagattggagagatgcgctgacaggagggaaggcggggatgcggtgaggggagaggcaaggggtgaaggcacagacagggccaggggtgctgtgtgaagggagggaagggccaagtatggggtcttaacaggtcttctgtacttacgggggcaccacgagctccagtgggaccagcagggccgctgggaccactttcaccctgagagcaagggacaagaggctcagggtcagggcctcccctgaatactcctagtagatgaccccaggagagcctcccctccttctggtccctccaggttcccaggttgacagctcagtttggcaggacctgctctcccaacgcaaccccacggaaccgtgcccaggcctgctgaggaggctatgtgttagggcagaaggtggggaggcggccacctcaccttgtcaccaggggcaccagcagggccaggaggaccaatggggccagtcagaccacggacgccatctttgccaggagagccatcagcacctttgggaccagcatcaccctaaagacatggataagcttgagatttccagtgtggggcaaggaggtgacctatagtgttctgcttgtgtctgggtttcctgagaggccctctgcagctctgccctgcctccacctgggccaaggactcaaagattctttcaggaaaaagggtgcctgcctggagactcaggtggaactgaggttaagaggcccctgacttgtgtagcgggatgaataagggagcctccaccaaggagatgcccccacctctcatcaccatggtaacccatggggatagatgggcctggaccacagggagaggattcatcagcaaaaaggccagaggggaaaggggaagaagggaggattagcgagaagagggacagatcccagagagaaggagagatgctgagagattcaaagcaggcagagatgggagccatgtagggctcaggggagggggaaggttgaacttactctgtcacccttaggccctggaagaccagctgcaccacgttcaccaggcattccctgaaggccaggggcgccctggctaccgggagctccaggggcaccagcatcaccctatgtgacaaccaagaagactggagtgaggcctggggccccaagagtgggtcacagccctaagccccgcttggtatctcccaggcttgtttccaaggccaacgacaagccttgagaaaagatgaaaagactcacagcccagactccaggctgtgtgtgggcccctgccctggtcttttccccccaccccacaccctatctccatggctttggtcatggcccgccatcccctgctgcaggaggggtgagacctggtccctgggccacttgccagagccccttccacgctgccctcaccttagcaccatcgttgccgggagcaccgttggcccctcggggaccagcaggaccagggggaccttgcacaccacgctcgccagggaaacctctctcgccctagaagggaaggacagggcatgtgaaggctgctctggagatagggccaagtacgacgcaccttgacggatgcagcgagagaggcctacttactcttgctccagaggggccaggggcgccaaggtctccaggaacaccctgagggggagggagagaggaacagacagtgagcaaaacccacctggggccactctgctggaaagcacggtccttcctcctggggtctggccgtgattagagaggaaccccttctcagcactgaattgagattatcccaaacagcccctctcttcctcctagggatgtgtcaaaggcttcccccctccccaaactatggaccaagatttatcaatagaagggttgagggaaagtcacagcatattccagcagctcttaaaagaagtcacccagactagcaatcatgcagcccccacttccctctgtgctgctcccttcatgggaggaagttctttccggcgtctaacctcaatccctctagttgatggctgtctgattagctaggagcgggggcctgtccctctggattccctgcatctcccacccctctggccggctgctccctcttacctgttcaccaggtttgcctgcttcacctggaggaccagcaggaccagggagaccctgtaggtgggaaatgggggaagaagggagggaaggtttagaatctggaagagaccaaagaggtgtttcctacccctacctcccagcatcctgacagccatgaggcctcacctggaatccgggggagccagcagggccttgttcacctctctcgccagcgggaccctgcacagagagaacactacagtcacggggaggccgaggagacgaggggctgagggtgtctccccttttctgctccccagatctccccatcagggacactcacagcagggccagggggtccctgagctccagcctctccatctttgccagcaggaccctgcagggagagagcaaaggggaactcagggttaggaggccccgagcagctgaggaccgtggcctctagcacccctcctgcagggaggagaaagtgccggggcagcaatgggaaggaggtagggatggaaaggagatacttacgacagcgccagggggtccgggaacacctcgctctccagccttgccgggctctccctgtggagaaagggagttagggttgagggggctgaagtgagaagccagggcctcctggggcctctcatttactctgagtgggactttttaagggactttcttttcaaaagactgtttggcccccggggaatgcccctgcaggtgcttcttgctgggccctttctgagcccactcctcccaagatgcccttccaggctcctctgccttttccctgttagatgcccgctgtggcctgcactgcaaagggctttcatcttctctgctatctccttttggacctcaaagaatccaccaagactgggggtgcctatcatatcaaaggcctgtcctatccccacgtgtcggggtggagggccaccaacaacacttatttccaggacagagcagtggtcatggagcctggagaagtgtttcattttttctttctttctttcttctttttttttttttttgagatggaatctcgcactgttgcccaggctggagtgcaatggcgtgatctcagctcactgcaacctccacctcccgggttcatgcaattctcctgcctcagcctcctgactagctggggttacaggtgcacaccaccacacccagctaatcttttgtatttttagtagagacgtggtttcactatgttggcccgactggtctcaaactcctgacctcatggtctgcccgccttggcctcccaaagtgctgggattacaggcatgagccaccgtgccccgccgagaagtctttcattttacagatagggaggctgaggtccagaaagtgagagtgagtggccacacggcaggtcagcggcacagctgagcccagagcccaggactccttcaagtctcaggtgtgtttgtccctggctcttcatggatcctcacttaatactcacagcagcacctttaggtccagggaatcccatcacaccagcctgaccacgggcaccaggtgggcctgggggtccggggcgaccatcttgaccggcgggaccctaaggatgggaggcacgaaagcagcagtgaggacagcagggaggcagacaggacaatggcagggggttcagggggagtgatacttacaggggggccagttttgccatcaggaccagggctgccagggcttccagtcagaccctagggaggcagagaggtatgagtgggacttggggagaagcatgatggaggtgggggaggactccagagggcagacccttgggcctgatccagaacgcctcatcccagaccctacacgggatggtcagggcctggccaagccaggctgaaagcctggggcctcaccttggcaccaggcagaccagcttcaccgggacgaccagcttcaccaggagatcctttggggccagcagggccaggagaaccacgttcaccagcgggaccctggttgggggaagtcacaggaacagttagggtctcaagtttgtggctctttgccacgggccaaaagaggaagaagatgcccagggagcggcagggtcagccccccggccgcaaggagaggttaccttgggaccagcaacaccatctgcgccagggaaaccacggctaccaggtccaccctgcaggaggagaggaggccagtgaactccgcgacacacaggcaccagccaggcaatgagggtggagcgggagggggcgggcagggacacttacacgctcgccagggggtccgggcaggccagtgggtccgggttcacctcgagctcctcgctttccttcctctccagcagggccagggggtccttgaacaccaacagggccctggagagggccgagaggaggaggcggcctgtggtgaggggccatcctgtgccagcctcagagccggctgaggctgggcctccagtgtcaggggttcctgggggtgtggcagggactcccccagaagactaggggctcctcttcctttctggatttccctcagggggctcctagggcagggtgggctgggctgcaagaaggatggcggggagacttacaggctctcccttagcaccagtgtctcctttgctgccaggagcaccaggttcaccctgcaaggggggagaagaggatgagctgagagtcgggggcgctcagttggcctgcgtcttcctgctccccagatgagagccgcactggagccagtgcatggggtgggcagaagggagagtttggtactcacgctgttacccttgggaccaggagggccgccggggccctggggtccagaggggcctcgggcaccagggaagccaggagcaccagcaataccaggagcaccctgtgggaggcagacagccagggcgtgagcctaggagcagagggaaaggggcaggcaggctgcaggcggcaggagtgggactgaagcctggcaggatacttacattggcacctttagcaccaggctgtccatcagcaccagggtttccctgtggcacagagaaaggagtgtcagcaacaggcaaggactctgaggttagaaagtggcaaaggggacactgagtcggggacacttacagcagggccagcagcaccagcagggccaggggggccaggctcaccacgcacaccctggggaccttcagagcctcggggcccttggggaccagcttcaccctgaatcagaagaaaggacatatcagaagccaccctgggaaacccaaccttgcctctcgggatggcaggaggaaggggagacagggacaggagagcaatgatcaggactctaagatcagagacggagaacccaggacaaaggtgttagtgaacgggctgctctcttgccactctgggtccctttggtttggggaacagggagacatgaaccccttggcccatgagggtcatgcttagaggagagtggggggtctcaccttagcaccaacagcaccagggaagccaggaggaccagcggggccggtgggaccctgtgaatgaaatggagatgtcagcgagaaggaagagatggcagctgcaagtcacaccctgggacagaggaagtcctggggttcagaccaacataacctgctcccattgtcagccccaagagcagatactgagacccctccccactcccaggccctgaggcctacaggccacactcacagggggcccggcagcaccagtagcaccatcatttccacgagcaccctgcaggagagaggggaagccccgttaagtccactgagcactggccagtccctagagttcctggggagccccttccagagctcagggatcccccaaggggccaggagtacttacagcagggccaggggctccagggcgacctctctcaccaggcaggccacgggggccctgacaaccaaaccaagagaagtcagatgagatgggagacagccttgttcccccaggcctccatcttgcccctgcctcctgccccatccctgccctctggaactgggcacactcaccatctgaccaggagctccattttcaccagggctgccaggctcaccctgtagatcagagaataatgagtgagaaattcattcatggtgggactctggggatgtggaggaccatgatgttcagacagcctcttaccttaggaccagcaggaccagcatctcccttggcaccatccaaaccactgaaaccctaaagcaggaaagaggtagaaggtaagaacctgtggagggggtggaacagccttgacatccacctagatctgagaaagagccttgggaggtcatcaccgccatccctttgtttctgagctagactaaaccccaactggctctagatctcacccaatcgtttaatcgggactgctttcccatggagggaggtgcttttggatgtccactctctgtcccttgggacttctgtagctgccacccaccatgatgcaactcagtatcttttggggaagaggttgggactggatgggggtatgctagggacttggggagcttaaatgactcaaaggtgactcactctgtgtcccttcattccagggaggccagctgttccgggcaatcctcgagcaccctggagagagatgaagaagacaaggaagggccattagaacacatcactgtggacccagctcctaaatgaagcccaagtgcagtgaagcccaggttcagccacagccccctgctcacctgaggcccaggaggcccacgctcaccaggacgaccaggttttccagcttccccctgagagggagagaaaagaccatcatgcctctgcctccccaccaccgcctaggggctggaaaagtggagaaggtctcagtcttctttggattgttgcaggtcaggtttgggggcccagaatcttatctttgaatctagagctcagtttaaatctacagtgaaaccttcgtttggtgaaatctgagtgcctctaaaggaatttctgtctacttctgtcaccagcattttaatttcaccatctgcgcaccaatttccccaaagggatggggttggggattgctctaaggtggctggggtgaggagaacagtcactttgttggggacatggaatcatctcactaagcgaaaagatcattgttccagggcagtccgtgcatcagagaggacttgccctccttcctctgagtatcgttcccaaatgtggtggagtggaaattgcaggacccaacccatggaggccatggggtcagatggtatcttcttgctggggatacttacatcatctccattctttccagggggacctgggggacctcggggacccatgggaccctagaaaagatagaagaggtggttagaatatggataagaaaaaaagaaggggaaggctgggattgaagggaagaggtaaggaagaccccaggcctgggagttcttctataggagagtctgtgtgtttgtagaaggagtatgaatctgtatagagagtgcttactgaagctccaggctcgccaggctcaccagggggaccttggaagccttggggacccttgagaagaaggaaaaagatgggttagaagacaagtccctgtcaaccttctccaatcttaccaagagatctctgagcatctctcctgccctcatcccagtcttccctccaaaagaccaaagcccaaggaggcatatgaagacgtcctggatactcacaggtgcaccaggggggccagggagaccacgaggaccagagggaccctatagagggagaagaaaggggggtcatggtgatccctctgtaggaaagcatgtggatgtagtcattcatgcctgttgggaccacccagtcgtcctgcatcctttctggactctgaggtcccaaaggtgatctggacctcccaagctgtctataccagccgcctatgcccacctcctctggatacccattctctcttctgtcatccatgctccccctgctggctcaccatggggccaggcacggaaattcctccggttgatttctcatcatagccataagacagctggggagcaaagttctagaacaaacaagagaagtcagagtgaggacagtgaattgaaaggcagaagacggcactgaggatggaagaaactgacatcatgcacttcctggtgtgatgtgctgagaagaataacattgcttttaagacattcctgcaaagatgcatccctgcatctaatctaatgagacaatcccaaattgagggacagtctgcaaaacaactggactcatcaaaactcccagtgccactaaagacaaagaaagtctgaggaacgttctacatttgtggaaactagagacatgatgattaaacgcaatacataatcctggactggatcccagattggggtggtggggggtggaatctataaaggacattatcgggacatcggtgaactttgaatacggactgcacattagagagtgaatttaatcattgtgtaggtcagttaagagaatgctcttgtttttaggaaaaacacacctaagtatttaggcgcaaaagagcctgatgttagcaacttgctcaaacagttcagaaaaaaaaagatacatatacataaacagagactaataaagcacatgtcacaaactgtgaagggtatgtgagagtttcctgtaggattcttgcaacttttctgtaagtttgaaattatttcaaagtataaaattatgtcatctgccaaaaaacaaaaacaaaacaggggagagtggacacacaaggcctctccacttactcctccgaggccagggggtccgggaggtccggggggtccggggggtccgggaagtccaggctgtccagggatgccatctcggccaggggggcctgcgggtccctgcagggggagagggcggggccggggtgagcgtggggccgagagccatgcccacctgcagccccccacagcccagagtgcaacgcttacccttgggcctcgggggccagtgtctcccttgggtccctgtgggattgggggagaagaaacaagaggccaggttagagaagggaggactgtgaggagtcacgggccgcgcaggggcaaaattcgagggcaggagattacctcgacgccggtggtttcttggtcggtgggtgactctaggggacgaagagacgcgcgttagagccaaggtttgctaatgctgctcccgtcggcagaggcctccagcacggagggccagcgagcgccgaccacccccagccccagccccaggccccaggccccaggccccaggccccaggccccgagcgcagccgcacctgagccgtcggggcagacgggacagcactcgccctcggggacttcggcgccggggcagttcttggtctcgtcacagatcacgtcatcgcacaacaccttgccgttgtcgcagacgcagatccggcagggctcgggtttccacacgtctcggtcatggtacctgaggccgttctgtacgcaggtgattggtgggactgggacaggcggaagaggggcgttgtcagtagtgactgcaacccccagcttaaccacctttcccccgggtctaagaggcacacttggatttttcacttcccgcccctccccccgttcttctttcctcaaatccttaaaagctcgcctgctcctcatcagcgcgggtgacagcgccgaggcgcctggccagggaggctgtaactctttccagttctcaggaatttaaacaaagctttagtccgcggtggcttccaactccaacctcagcccattggcgctgaagccaagtgaaataaaaaccacagggtgagagggggagggcggggggagaataggaggggccccatcgggagggcaggctcgtggggagtggcttgcgtggtagagacaggaggagggcgagggaggagagaagggaggtccagccctcatcccgcccccattccctgggcaggtggggtggcgggggcggggctaggagaatgggagcggctgattggaagggaggcccccccaagacttgaaatgtacctcaaaattaggagacaccctaacctccagcacctagacatcttaaaagcccgctctcggggccatccccagtcccccaaagccagccaatgcctccaggtcacttttagccaaagaaccaaaaagtccgcaaatagtccagaagttagctaaccactcccaccgcgctagcggctccccagacggccactaggtgtcgggcaacgctaagcaatggggccggggcggggccgacctcaccggcccgactacctctttaagaggcttgggctgccccatccctggctttactttccagatctagaggtgggggtccgcacccagcaataacccgggtcttcccgctccggctcctgccccagtaagcgttggaccgggagacgcagtgctcagcatcggtcagcagggggcgcaaggaccccgccccgccgagtccgcgccaaagtttctcatcctccacccgcccacgctccgcaccccctccgcggctgcccagcacccccacggccccagcaacactctagcccgcagagagggcaatctttccttatgaatcatcccacggccttcctgattgcttctacatcttgggcttcaaatgccccgctcggcgcccccatcccatcttcgacgtgctttcctcactctgcacccagatccccacttccccggttgcgcgctcgggattctctcttgcaccaactcacgccgcgctccaccctcacctcccgctcccggggcgcgttcaaccccaacctcgccccagggtgagctccctcctgtctcagggcgccgaggaagagccctcatcatctcccttccattcccgagtctccggatcatccacgtctcgttttaagccgcggcccccgggaccaagcgcaaggcgcgatatagagtatccttgcactcccaaaagtttgggacttactgtcttcgtcttggccctcgacttggccttcctcttggccgtgcgtcaggagggcggtggccgctaagaggagcaggagccggaggtccacaaagctgaacatgtctagaccctagacatgtagactctttgtggctggggagggggttagcgtccgctcatgcgtggcctcacactccgcgtgcctcctgctccgaccccgaggagaaactcccgtctgctccgacgactggcccgg

SEQ ID NO: 4 - DNA, 36672 bp, Homo sapiens
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatgtaagtgccttcagcttgtttgggggagactgggtagagaggttagatgggagggcaccctgccctgaaaaggaaaacctgtaacctgaattccaggtacacttggagggcagactctcaggcatgtgggaaaacgccggaattgataagaaacatggaaattactttaaaaaatgaaaacataaaagccttgccaaaagttagggaacttttcctctaagttcagagtgagacagttaactcggtctggctcctcagcttagtaacccccaaagggagcggaaggtctttttccctaaggatgagatattaacgaccaatgtggtggaggaagtcaagggcctgcaccccacaggccccataaccgcactgatgtccaccttgtaaaacttgaggcctgcgttagaaagcccttcaactgagtaatgtaaaactcacctcctaagagcttttatcttctgggcattgtaaggcttgtccggaggaggaggatgacgatgctgatatgatgatggttataaggcgccctctggaggaaggaaaatgaaagtacaggggacagggccttaagcagatggaatcccaattaaagcttctacggatttatacagattaatgatcagcatttctggttggagcctttcccagtggctagtcagtgaaccctggaaagaagaatggatgctacttggagtgggtacattctgaaaagtaatataagtgtctcaattcactttctagtcatggaaatggtaacattttttaactcaaatctgctctaaattttgtttgagcctgagaattacccctttgacatgttcccagtgataagcaaacattatgaacgcagcaagttgagaaatatcaacattgagatgagactcaagagaccggggtttttcccatgagtctgacaccaatttgctgcgtgactttgggcaagtcaaacggccttttctaaaatgtgagacagagattaaagggaccccaaggccactttccagctctaggttccatggccagactttcatgtcaacagagaatgaagaagatcagtccgttttcatcttgaaaatggctgccaaagtgctagacaaagatattgactagatgggggatggtattgtctgaccacacccagtactccaaaaagttgttccacccacacagcacggtgtctaccactgcataatttctaatgcatttgtgtgcttgtgtgtgtgtgtgtgtgtgtgtgtgtgtctgtgtgtctgtgtgtctcttcccccttcattcacttttagtatacatactgtggatactaaggagtaattgcagtgaacaaattcacattaccgagttcatatttttaatgagatcttgagagtgggaggaaagagtcggctcctagagaataaaatgaaggcagacttagggaaatttgaaggtacaaaggcaacttaccttctgatcaacagccaaccacagtctggaataaatgttatcaaacacacattcttcaaaatggtccatgtctgagtaattaaaaggcaaatttccaaaatcataaggacttccgttaatcaagtcaggcataattattcttcctactgatggacacaatgaagtaaacatatcattcttgtaatttaacagtaattctcgtaaattgcccttaaatgtcagtgctggatgtggtccaccctcctaaattgtgactgttgcaacagatgttctcacttcaaataacgcacttcttggccacctaattaaagcaatttttggggtgattcatcctactgcaagcttggccacacttgtatcctgtattaacctataatttttgtaccgtaggagaagaattcactctttaaggacttataacaattatggcaaaaggggggatagtacttttgtttattttttctattatttttcaagatctttaatccggtttttccatttatacaaaactctttctccgagacaaaaatgatacatattggtaaaatgatcttacctaatttaagtgaactaatttaaagcaaaattcagatgtctgaattaatccattttcatagttaataatgtgcaaattagaccttttgaaaaaggatattaagaatggtacaaactcaatgaagtactaggtaacttcaatgttttataaaaaagtaagtcagcttcaatgtttcataaaaaacaaattcaatatagaattttaaggtaacatactttcctaaattttaccttttttcgatatttaggtattaaaaatgatcaaaatcataaattatttcctcatcaatctactagtcttacattcagcgattcatctgtgcactttaccagcttaattgctaagcattcaaaatatccttcagacacattaatatttcacaacagttataaaatagtaaataattaataatttaattcaaaatacatttacatattaatattgcaaacaaatcaccctgctgatccctgccatacttttgacctgcataatttctaggtcattaaaatattcttaaaaaaatataattggtccttaattaggtaattcaattctataaacttgtttctctatttgttaattattgctattgatccatgaagtgatactaataattgtttcctactttttcttttttttttctacagctttacaagaggtgagtaaaactttttttagaatttttaaaaatactttgattcccttggctacagtgatgtcttctcttggaagggaagaagttacattaatattgaccatcctagattcccaagaaaattgtgaagaattatgatagtcaaaacctttctggctgccttagaaagtacccacccaattttccaaaataggcggggctactgaataagactaggtttataaaatattcataagaaatatagagtaaataatccaatagaagtttgagttttaggatcagcttctatgaagcagaagatttcactgagctagagaatcttttcactcctttggaattatttgcaaaagcacttattgttaacacattcttagctcatgagttgaatttgaggcataagtacaggtacgtattgctatgtattttttgttctgtaggtacatatttttatttgacatgttggtaaaattttaaattgtagtttgaaatattaaactgagataatagtaaatgcataatgtaatgaattgtgaaggtatatttgtatactacaccaaaatggaagctgtttttaaatatatatatacaattttcttcataataatctttgatttattcttttctaggaaactgtaagaaaggtaagagtacactacttctccataaatatctaaaattatcagggataacataatttaactaaatttatagtagactatagaaggaaaatactttatcaaaattttgttcatatgaatatacattagctaaagcataaaataaagtagctttgatgtttaagataacaaagtttaattatcttctggaatcatctgtaattacatttatgtgatacaaactggtgatttacatacaaaaggaaaaaaaaagacttgtttttattctggagatggaaggcatattatgttaattatagggagtaaaaaaagtttattttaaagggctttgactatataaatgtgctgttaaaaatgtaacaaaatgatcatttaatctacagttatcatcttattcaaaatgctatgcatagtattgacctaatagctgaagactatagcagcttccaatcctccagctgaaaaaaaattacgtataattacaattaaaatatatacttttatctattgcattgtgtcaattttttatatgctatctaataacattgtagttacatcagtcttaccaactaattattatcaagaatgatttgtttgttcactggaaattacttcttaggcatttattattgtcctgtttgtatctttcctgtagggcccagccggagatagaggaccacgtggagaaagggtgtgtaatttttgaactataaagggcttcgtcccgtatttgaataactatatgttagaaactacaggaactggcaatttataagaatattatgtatccagataattgtacacccctttaaacaggtaatgcactgcagaagaagcgaatgagcattattatatatgatcaatatttgttttaggtcaaaattaccgttaaaaaagaaaaactgttacagtcatattctttgcatggtctactttctttatttgtaattgacccatccaacacatgcataatggaaatatatctacctaccaccacagtcctctttttaacacatttcatttgcttttgaactaagatcccttaggtagcttggaaataatagtgaattagtagtcagtaacatgtttctctgctcaaattcatgcatgtacaagtcaggcttacattttatttgtggcattcttaaatctccctgctatgcttatttgacatttataactatgtggttttgcattgtataacacttttgccaatatatgaatacctatatcttatatctattaggaagaggagacttacatgtatttcactcaatttattagaaatagaattaaatcagttaattattttaacaatacaagtagttaatgatagtaaatctgcaggattttctctcctatgataaagtgaccttattaactgtcacatcagttaattcattcacatgtaacataccaaaacaattgaatcagtttgtcacagtcagagatcggcaataaaaatacgatgtaagtccttgtgcactgttaaacatatgaagcacgtggaaccatacattttggctataatttttatatttgaatactggagcttcagtatgaattaatattcaatggccgagatagttctttaggaaaactaccctgtgatatcttaagagttattaaccctctttctaaaatagactcataagtgaatttcaatcaatgacaaatatagtatattaaatttccaccctacttgcacatagaaaggtctgaacaactgatcttaccacatataattcttaggtttctacagggcctgtctaacctgaccttactcactttttacataacagggtccaccaggccccccaggcagagatggtgaagatggtcccacaggccctcctggtccacctggtcctcctggcccccctggtctcggtggggtaaggtgtcttacgtattgctaacttttagctaacttcagttgaaagaaggtttattgtggaatttatttttagcagttaagggataattcttccatttgaaaattagtatattttatttcatttatttggttttttcactcaagattctgctttacccattctctttgtgagcccttgtcaattacaggactggtctttgtgtgcactgaagttagctgtggccatcattaccattatttaatttggagatttaatatcttttattagtaaggcacaaataagaggtgttgcattattaaggattttgattagattgaactgtgtaagtgaaatccctgatcttaagcaattttacaaacatcctacgctttttattctccttgacttgaagtctgctgaaccaacattcaaagcggttttaggtttaatttgcttgaaactaatttgagaaaagtacatttccctttttcattaatatcttcttttagcttcatgtctttaacaatggtatgagtgccaaatgacctcactgcaggaaggaaaacatatttgcttaattggttagcactatgaatcagaagcctgattctaatacccaactgtatgtcagtaaaataagacctttctcccccaaagatcttattatgattgcttatctatgaattgcattaaaaagcagcttctttaatagagctaccactataagagagatctttaacagtaaagttattactgtgaactagtttttagaagttttatcttccaaggggtattttaatttaattttcctctaaacttgaaaactctttatgcccttcctgaaactccagcaagaaaaagatctcttagtcattttgtgtagctccggtggggaagggcaacaggtgaaaatgtgaagatgtcctcttgagctctgtctaatttgtcaggagcccttagtaacattaaaagtttagaaagcttcccttcctcagagtagaggtaaaaggtgggagtggagacaccgagttaaggcagaggaagggctcaaaaagtaaagtagggaagttctccatttcaaagaggtgtcggccaagtttttgacgtacagctctcataactttttaggaatttagttcaatatagaattttaaactaataattatatcaaaaacattgccctcttttaaataacaacagaaaaatatttacaagtagaatgagaaaatgaactacatgactagtaactaaaaatattttatatatatatataattttttttttttacttctctagaactttgctgctcagtatgatggaaaaggagttggacttggccctggaccaatggtatgcttatctgtttatcttagccaaaaaaattgctaaataaatcattcattttatgtcacattttaccacgccatttatttagctacctaagttaacactcaatacttagattatataaaaacaactctttttgttttcaaatttatgaaaacataagttaaggagttcacttttctttacaaaagaaagattaattgatcttttatgattatatgatctttttgattatatgatcctcattaagatagatcatatacttatgtccaagaaataatctttggacatagtaaccataacttgggcaaatcaatttaatttaaaacagtaatcactctgattaattttttaatattctttaacattgcttagaattttaagcaacacttagaggcatagaactatttattaagttctctgaacttgttggaaaggatcaacaagttctatctagtccagctaactcattttaaaatgggagagtttaagcccttttctcaaagtcatccaggtaactaatgacataactagaactagatgccaggcaagatgtctaatatttgcttacatcatggtttatgtacctagtccttgaataaaccactcatttagtcaacagatattaatcagatgccttcaatgggccctaaactgtattaggaactggggaaattacaaggaatatgacagattctgatccttcctcaaggagttaacaatataggaaatgtttctttttctgaattttgaccaaaaaaatccttttttagtctattgattgtaaatctatatagaagagagtatgagtaaaaatctagcatttatgtcactcagtacaaatattcagcaccataccctatcagtggagcaccgcttagaaacattccctatatgatgatgatgatgatgatgactattaacaaatgaagcttctaacaagcattagagagaagtttgaagggaaaaatgctaatacgagcatgcaaaatgtatactagcatatatgaaatagaggggaaaactgccagaagtcaaagtgttaggttgattaagcactacagaatttaatgtatacacacacacgcaatttagtgattttaattaattgtttcaaaacaaaggtatttatctgcccaaagtcaacaaggtctttaaaatgtaaattttacctgggcagtgcacttagtgctctatcttcaaaagaagatgttctgctggagctaatggcccacagtaagctaatatactctaagggtgagataatattttctgtaaattaaaactcccacttgagaaataatgtacctttaattgacgacttctaattccctaattttttctggtagtttaaaatgttcatatctgaaatgaaaaagtagagtgtttcttttggctttgtttatattggatttttgaaattagctgtttcagctaatgctggacattagtcagttttaaagcagtacctacatctcaagaagaagcaagggggcggaaagtaaagagctactaaatgtcatttttaaaaagcccactaagctgggaaaattaatatggatttcagatacccctgttttcggaacatctgtcttggcataaagcagagtattttactttgaaatatcagtgaaatataatttaagcttgcacatccacacacatgcacagacatatgtaatcaacagatatctgtttcacaaatagggaagataggcagcaataaagtattaaaataatttccatgttggaaaatcaataactataaaaccccacagggttcttctctgaattaatgagtaatcacagcctccatgaaatacactacattttatgtaaatgaaattgttgcaaatacatgaaaaaataaatataattagaaattcatgatgtcaaagaaaattattttttaatgtatgcctaaaaagctattgtgatggaaaagtgacagtttcttttaatgtcagagcaatttctaaaaccaaatgaataattcttataattaaaatgacatacattttagataaaatccatgttatttcactctaggcattaatacagtaaggtaggtttgactgcagagtccccacagctgatgtcatgaacaaattacttgagactggtacatgaaatattttcagcattatgaggaacagaccctacggatgagcttacacaggcactgattactgcaaagaggagtcaagaaagtgtatttagcttacaaactattaacagcctgttttaccctacttttgtgctatggaaacaacaaaggggaaaacaatcttccatcatttgggccatattttcaacaataatatcatataatagactcttccagaaggctgtttcaataatgttttatttttccttcacccctcattacatccacttttgtttgacattttcatcagtcaccaataacccttagaggagcgataaggttataacaaacttctctctaatcattaagaaggacttttgattcttttcaatttatgtcctttgtggcaataaaaataccaatttcttagctaaatatgacataggaagatgacatatgatcaaagatatccaaatggacatgcttcatctgctgtatagaagacaattgtatattctgcacttctgcaaagactgattcacttcattgcatcagaacaatctcaatatgcccaattgtgcacaactttaaggaacctatctgccccgtctaattctcattgatttctgttgataaggattgggagaaaaggaaaagcaaagggagagaactagtgcaggaagtttgagtccttaaattcttccttgggaggaataaaaactatggaatcaaaccacaacaatggcactgctaagttggtcatatctgaccccagccaacaccatgacaacttatcagtgctaactgttgatatatctgctttctttacagggcttaatgggacctagaggcccacctggtgcagctggagccccagtaagtactgaaagcttgtaatgcctcttatgtaaaaagacagagaattaagaaataaaggcttggagtatgacattctttttttcttttagggccctcaaggtttccaaggacctgctggtgagcctggtgaacctggtcaaactgtgagtacatttttccacctttgtgataagtttttttccaggaagtttatgaatataaccttagtgaaatgatgggtctcccattttcttagggtcctgcaggtgctcgtggtccagctggccctcctggcaaggctggtgaagatgtaagtatttactcttaagcactttcaaaatgctatttaaatactcttgcctcaacaagattttctagattcaaattaagtattctgccaaaagctgaatatgcctgacagaactcttaatgtatgggaaatattattttaatgaaatattaactaacctacttgtattaagggaaagattaaatatatatctggatccatatttttatgtgataactttctccccttttgtaaaaaccaagattcccccattttgtctgatagtttaccaagaagaagttgactctacaatgttttcatgtttagggtcaccctggaaaacccggacgacctggtgagagaggagttgttggaccacaggtgagactttttacattggtagatagcacaaacatcataggcctataagatagttgctaaaactagcatcaatctaaatgacaacatagatgtcacccaaactcataacatgaatcgaaggcatctaataaagaaaaaagcctagttaaaaaaaaatgcatatacattttattcatgcaaataatggaatataaatgacagcaagcataccataagcaactaaattgtgttttctacaaataccgtattattagttactcacattagagcaagttaatttgtcactctgtgcttagaggtatactagactttggttcaaagcttgaactttgatgagaataaatactttggagggaagaagtcactgtctttttatttatggtaaaacattattcaccatcttctgtatttctttctaagggtgctcgtggtttccctggaactcctggacttcctggcttcaaaggcattagggtgagcacattctttactcagaagagagaaaatgcctattaatttttggaaaaaactcaagtatgtttaaaatcttgggtgacatatactcactttcaaatccctggagtttgccaaagggaagaaagagttaaagagtcagatttcttgaaagtaaagtggggtgcaattttttcagtctgttcatagctaccaaaaaacaggctcactacagagaaaattatatagaacatgtattacttattgagtatttacaaccgtctgaaaatcataaaattattaaggatggaaaagatgtgagagaacacctagtcctccatccttctctctcaatggcaagaaagttaagtgacctatctagggcaatagactgagtttgctgggacctggaacactggacttctttctactgcagcagacaagacttacccaagagagattaatggcaaagatatacaatacaatttttatttgaccaaacactatcatggaacagcattttataataaggctttcctttcagggacacaatggtctggatggattgaagggacagcccggtgctcctggtgtgaaggtaaatattaaattagaagcactgtttttaagcacttgattgaaattccccatgacctccaaaaaagtatattatactgaagactacccatattacaaaaagtatttttattttttttctttcctgtacttcaaatccctcaaggatggggactatgagagtctgtgaaaaaaggtcaattattaatatttattaaaattcaatatctattaaacaattgagataaaaataatattaatagtttcttgttccatttcctttcctccctctataattccagtgtatctctgcagccaaaataaaagtaaataaacatataatcagagattacgacactctgtattattttaaactgtaaattctcctttgccacacactaattagataggtacattcatgtcgctatacacttttcaacctctttcctgtgatttatctgtgcacactcaaaaaaattttaattaggtaattaaagtctcagaagtgtgttatctcttggctaggctcttctctgacagcgttttcaactataaaatgttctctttcctattaaggagataatgtgatattaaagtgaataccaacgtaattacaaattaatgagtaacgaatactagcgggaccagaaatgaacatgaatatggagaatctattctaactttccagctgccacacaaatggataaggtcaaactcattctcccaagagcccgatataacagctcagactactaatcactgtatcataaaatgttagagctgcaaggagctttggagaccccctcattttgcagaggtgggaaactgaggcttcgtgagagcaattgacttgcccaaagtcacacatctagaggttagaaagtcataggctagaaatgatccccccttgccacttcaatgcttattcccaaagaatagacttcacatagaatcctggaaattaagggtccttatgaggtctcttaaaccatatttcccctatatctaaatcagattatctttaaaaaaagttcttttacatgtgccatagtattaaatcccactactactactactactaccctggtttttactcaggataagaatatagattggaaataaatatgatggctctaaaaaataccatgaagcttcaatttttcatgcacattttatgaaagtgataacactgagtgttcaaaataactttaaaaaggataaatatggttacattgaaagcaaatttatcctttgccatctctttttatgatattgtttctagtatataattgatatcctgaatctaagggagaaattggggaggaggtacactcaaataaccacatctccttagaacctggatatgtggtactatctgaataaaaactcatgttagcacattttaaaatctgtgtgtctggcataattgaaaaacaatctatatgtgtaagaaatattatgaagtatatgaatggttcaaagtaaaaaaaatagagtaaaattgcactatcaggaaaaataattgttatatttaatgaacaaaaactcaatccttctccatgtagggtgaacctggtgcccctggtgaaaatggaactccaggtcaaacagtaagtattgactacttcattgtaaatttaaatgtgtacactctttatgagatggaacttctttaatgtttttgctaatcactgtatccttcagcattgtattctttgatgtttttctaatagccttctgatacttaattgaaatccactactgtttagttggaattagaaggcaacttatttatttttagtgtattcttgtacaggttggaaactgaacaaagcaaatgatgcctgtgactttttttaaattagcattctggattttattgaaaatatttctgcttctagggagcccgtgggcttcctggtgagagaggacgtgttggtgcccctggcccagctgtaagtgcttccatttttgttcagtttcatccttttaaaaaatcttctaatggctgtcatttaagtttccacctgatcttccctttattttcttcttagggtgcccgtggcagtgatggaagtgtgggtcccgtgggtcctgctgtaagttttgacactggggagtttgaaaggagttgagaatgtggggtgggtgctgtcttcttcattaatctcttacgaaatagcatcatttcagacactttaccaaatgttctgtgaggtcttttgaaggctccatttataagtagtgtaagccatttataagtacctgaacttttgattgatgtataaagcaaaatatccccaccctggataccatgaatgtcttgcctttgatgagatcctaacgacaacagactggttgtcagtttttttctttactaatataaacagtgtcatgccactgtaagcaacttcaatcttctgccattgttattgttttcttaatttacttggaggaaatttcttaccaccttctgctttgatttcagggtcccattgggtctgctggccctccaggcttcccaggtgcccctggccccaaggtaaaaacactggtgaccattgtcactactttgataaactttttactgtgatgtgaaagattggaactgtgtttgcagataaagagataattacgaaacagttaccttaattattccttcccttcaaaatggacatagaatgaccagttttctcactctacatttgaaatagatcatttctctgcactgtgcactgtgcccatcgatatagatgacaacatggaaattgtctctaggactagttagttaggactgactgagaaccagagtcaaccacagagagacagaaggagagggaaggtagtaacagtagccaagatggcagaatcaagcaaggaaaataggaaaccaaactcaaatcttgtaataaaacggataagaaaaataattgcaattttgaagttttatgaagacatttcataaaacttggcatcttaaaaacagatatgctgtttcattatttgctggttaattccttggtttaatttcctcttttagggtgaaattggagctgttggtaacgctggtcctgctggtcccgccggtccccgtggtgaagtgggtcttccaggcctctccggccccgttggacctcctgtaagtagccactgtctttaaactttattgagtaaaagaaaacaaaggtggagtatggggaagaagaagaatgaagatggggtcaaagaagaaccgaaatattccaattaactgatatccttctcctttccttttcctcatagggtaatcctggagcaaacggccttactggtgccaagggtgctgctgtgagtatacctgcgtagctaaaatgtgctgctatgattttaaaggcatttaatgtgtgctgcctctacagcccatcacctccctaatggaccacactgcattttccttcacagggccttcccggcgttgctggggctcccggcctccctggaccccgcggtattcctggccctgttggtgctgccggtgctactggtgccagaggacttgttgtaagtggtcatgactgtggttctcatcatcctgaaataccacctctgccatcatttcatcactatctagacttccacttgtagttttattattcctatttttctcttccttagcatttttagtttatatttcttatatatatatgtacactcccgtctgctatatgcacacagacatgcccttcctgttatcttaaatcattacctcaaggtaaatgaggcaaagttctacaatatcagttttgtcccttcgaccaataataccattcccctgtactcaatttaaatatgaacagggtacatttcctagagaacttgagcttctctttaccttgacccacaaatattctaagagatttgtctgcaagagagtttcaacaaatgtttgtcctttgaccactgttctgtattgaaccctagggtgagcctggtccagctggctccaaaggagagagcggtaacaagggtgagcccgtaagtagctctatcatcacacttttataaagttaattgtttttctcattccagtttctccagctggacatagtattaaaattatttttttactccctcttcttttgttcttttcattaaacagggctctgctgggccccaaggtcctcctggtcccagtggtgaagaaggaaagagaggccctaatggggaagctggatctgccggccctccaggacctcctgggctgagagtaggtttcaaatgctcccaacaccctaacacaccagaggcagattatgataccccttcactgggaattggtcaaaattactgactgtgttttcttaggcaaaaaaagcatctgctttccatctgccttattaaatcagtgactctcaatttaatatgttataaaattggcctggaaacaatgttgacctacttttgcaggatgctcatctatgaattcctctaggggttgggtgaagtgttttggcttggtttgtgtctgtatctcccctgtaaggagatcatgctattttttacaaactctaccttatcaaagccaagagatttctttaattctctctatttcatgtactttcttgcagggtagtcctggttctcgtggtcttcctggagctgatggcagagctggcgtcatggtaagctgtctatcacttacttcctagaaaggggcttgctgcttctggtggtgggtgtgtcattagctttagcatcctcctcctctatctgttttttttttttttttgaatagggccctcctggtagtcgtggtgcaagtggccctgctggagtccgaggacctaatggagatgctggtcgccctggggagcctggtctcatgggacccagagtaagtttcaaactgattctgagcaaatcacacctggcattacttccttctttaaagggttggttaatattgaagataacaataaaaacatcaaaagtaaatttgttagtagtcttgctgacagttgcatttttgactttatcaaagctcagtagatattttcatgcatttaattagttcataaattttctatttattacttgatacaatggctatgaggtttttggaagaatagatctattttaatatatccaaattagattggtcctcctatcagcatgaatcttttatcttaatttgtgagttttatataaggtgttcatgaaatatattaggactatacatttttggtttattagattcataagtgaagtctttttcctagcaatcacaaagtgctgtaatgtattcagcatcacactagctatggagaaataacctctaggtccatagacacactaatccatagcaatagagtaatttttttgcctccattgcctcttatgggtgaatatcaactgtaattgtaccataaacaagtaatagggacaccaaatatagcaataagaaatccactttggaaattgtttactaaaagtattagtttttctattatgaggtaaataacgtgatacattttgcccatatacatgttgcttaacagtttcttgagatatctataaaaggatgagttgcactaaatttcaataaaaggaaagccacaaaaaaatagaagaaaaatttcagaactcttttcacacttcccagctagtggctaatattcctaatgatttaccctaggcaacaaacaaaaagtcgggggaaaaggtgcctttgttagacttcagttaatctaaggcttgagtatgtaagttaaagtgccaatataaaaacatcctcatttattttatagggtcttcctggttcccctggaaatatcggccccgctggaaaagaaggtcctgtcgtaagtattgctcattttccattatattttcaaggacacttattgcacccttatcaagtctattttgtggcttatttatacatgaacacattgaaaataaatatcagacacatacatcatctgggaatgcagagtaatagattgtaattatggagtccaaatgaatacaggactgaaagcagagcaggggagagaaaaacatggcagggaaaattgaagcaggtgacaaggggatgcaagagaagggaatgagggaaattgcatacatacgagattgaattggctatgtgtgtactgacatcctagttagaaaaggaaaatggattcataatttattaacgctttatacaagaagctctatgcattcagaaaactattctgtttcatccgtggcagcatcataagcttgaggttgtgagaatatgttgacactgagtaaacttgaaataactctgctttcagggcctccctggcatcgacggcaggcctggcccaattggcccagctggagcaagaggagagcctggcaacattggattccctggacccaaaggccccactgtaagaatcaccacaactttcttaccctcagcactttctgtagccaaattttaccaaactctagtatttatctcctgcgaatcagtccagtctcagggagtttcctttcaacacaggaaaactgcaggccacttatcacattaaaagtttacctctagtgtatccttatatccctgctaaaaatccatctcctgagccccatgcttccacagacacagggacatcttactgtacatggagctgcatggtgatggatcatccttagataacagaaaccacagactagggatctcaaaagaacacaaaaacaagcaggattcaacattgcaaaatcaccgtggttaatttgacattaaatgtgcaaagctgttctttgttttgtttttcatttttactctagggtgatcctggcaaaaacggtgataaaggtcatgctggtcttgctggtgctcgggtaggtgctaacttgtgtacagatctattcacatagcattcatctaagaaccacacttttttttttacaccatctgatatcattttgtcactttcttttcaagatggcatccccaggggtccttttactatcataaaatgcctttttaaaaaccaaacttataaaacagtgagcaaaaacaaatcagaatatacattaggtcaaaaatacagaagcacttggcttttattttattcattttgtaattaaaagggtatgaatatgtagtagcattctctggcctttataaattgccttgtgtcgcatacttcgcttgagtcatatcaaaagttagtaggcaaacccataaatatatatacctactatgtacccacacaaattaaaaatttaaaaagttagtaggcagtatttgggctttcgtgggaacccacaatgagtttaattcatgctaaaatgacaaacttgttttaaggaagtaatacctgaggctttgagacatcttaaactacctggcttgcagctaaccatcagcctttctgttaaatatttttagggtgctccaggtcctgatggaaacaatggtgctcagggacctcctggaccacaggtgagtatttctcccactcttgtgctcttctgcactagaatgtatatagtcctcaaactggccatctccattttcagtccaaaagttatacagctagacaacagtggtgacatacgttgctatttatgctctctttcctgtcactttcagggtgttcaaggtggaaaaggtgaacagggtccccctggtcctccaggcttccaggtaagtcaactcaaacatatacaatactgcctttggtcagcctattgagctgtaaatcaccataccgtacctctcttctccaccacaataaacatgatttcaggactgaagcaaagaaaggtgcatttttttcaaacaaacttttgtgtaatgcttaataacatacaatcgtgctcatgttgatatttggtagccaccacccccaaactcaattattagcaaatctcctgaacgtagccatgggattgagatttgtatttcttttcatttttagggtctgcctggcccctcaggtcccgctggtgaagttggcaaaccaggagaaagggtgagtaaaacaagtaatagtaagtagtaactactaaacttgagaatttccccctgtttaataccccactgctatgcaattataatatgtaaaagaaaatttcgtatttcatatgttaatgatagtgttttacatactttggtgctgatggagagaatgagccaaattactttagttctgattactttgttttacagatttaatgaaacatcaccttatgaaagtaaaatctatcaatgaatattttatttaatagccttactttttgtattgttcttgataacgttaagatacaaattatttccttccccatagtgaaaaagtaaatgcacaattttcaatcaaactagatcccgaaaaattccttttgtgtttttcttggcattcagacatgacactactatacacaatcagggcatgagttctgagtcattttctctctaattgtgatgaatgtgcccctatttagttacattctgtggcctggtctcctttgtcaacagtaggacatattaaggagacagctggtcagtaataaaagagatacacttgggtatacaattaactaggcaatgtacagaatatgataatttctcttaagaagacgatctgttatacagctaaaaataggcaatgtctaatattcattattatttatcttattattgaaggaaatagtctgtcactttttaaaaagcaataaactaaataatgggaaacaaattttttgataccaagttctgggatggatacatttttgtcaccaataaaattctctctttctgtatctttccatactaaaagttgttcttattagcctgtgtacttatgcactcatgtagatactgccaggtttatttcactctttccaaatttttcaaatattttaatcataagtgaatttagagatcacacacagatttcatgctttattctcatgttttgtctagggtctccatggtgagtttggtctccctggtcctgctggtccaagagtaagtgttacttcattaactttcataaactctggcaatgtgtttttaaaagtagtagtgctttctccttaaagccactgatgaccctgcaacaagtctctgatgctcttctatagtcaaatgtaatctgtagaaagcattagatttctaagttgatagtagaattttttgtttcattgctcatatttcctattcaataattacatagttataagaaacacataaatcaatatatattatagtcagtgatttatatagacaactatgctacatttgtgacagtggctcaacttgagctaaggaaaaataatatgttactaagatataaagttaattttggccatgtgtgttgatgttcaaagcctaaagccgaacttatgagtagtcatataaagaaaaaaaaacttagtttctctatgggtattagcatcactgaaatgattaatttgccctgaaagtattccatcatgtcactagttaacacatatgtaggaagctcaaagaaccaacttataacaaggtcctttgaaacagttacaacgtggacctatgtgataaatattttgggctatagaatgctatgctctagtgatatttagatgtaaattgggagatatttagatagacagacatatatatatatacacaaatacatatatcaaatatacatatgaatattgtaactgttatatcattattacacagggttataaaaagggggcatagataggagaatatctaatgttatctacacctgccattgctattcaactaaaatgacacaatcttttctttgaatactacacataactggcatctgtttttacctatggatttatcacagaaaagactcctcaaagaaggggaatgaattgcaaaaattgaatattatactctagaagcaacgaattctggagtcgtagtcatggaacattagagctaagtgacactttagagaatatctcatcaatctcttcattttactagtggagagattaggaacaaaagaaattttaatttgctaataaatgcaaaccagggctcggaagctacacaaatgtaaactctcatatgtaaaacagtatcactgaaagtgatgaatggtgcaacacttcttctaatcacttttttcagggggaacgcggtcccccaggtgagagtggtgctgccggtcctactggtcctattggaagccgaggtccttctggacccccagggcctgatggaaacaaggtaaaatcttatgttttctatattgctggtttggcccagtctgcctggaataagtagaccctttacaatagaaagataattgtttttcagatttttatttatttccagttctgtgatgacttccctctcagtaaacagcaatccgattccagtggacctgaattattctaaacaaacaaacaatagcaacaaactgtgggggaaaattcagagttcccaaaacataaatgaattagtatgggttgtcactcttttctcctcacgctgtttatgcttttgttttaatctagaaacattgtattcattggacattattttcagagaaaataactttttatcttaacatctcatcccatagagtaaaatttcaacaagtagtctgacttttaataataagagtttatgatgatgaaaattcattgggcaatacattcacccccaaaaatttgtctggaaacttgtgttccaaaatagaatctgtggtttagattttaaaatagatttaatatacattcctgaaaaagagattaccttaaccacaataaaaagaagacaacacatattattttcattcttaactctaggggaaaaaatgtaaacattagttgcaaaaagctattttagtgtatggaaggatgttcttgggaaaaaaataaaaacataaaagggagagaggaaataaagaaaccacggttttgtgaggtagtactttcaaagggatctatgtatctcagaagctagtcaacaggttttaagtatgtggaattgtagggttttatataaaaatggaagatacagtctctactcttaaggagattaaaacacaaacatctcctcaattgacaaggtctctttccatgctttctatctgggctaagagacttatccttgaaaaatgtttgtgggtaaacattttttactctctgcttcccattgtcctatcctcttctccatgcctgccatccttaagaggactgaagcaggttatagaggaatcgcagctgtgcactcccactaccctcatctcttcagtcaccatgtcattaacagcatctctctctgctatattctccctcctttcaatagcccagccttctttgtgtttcaaagcaggcaagaagcctgtctagctagctgtttaaattggaattcttctagagtttgattcttcattttcttctttctccactaaaattgatttcacatgtgtttgactcaagggtgaacctggtgtggttggtgctgtgggcactgctggtccatctggtcctagtggactcccaggagagaggggtgctgctggcatacctggaggcaagggagaaaaggtacgtgttgacccctattacatattgttgatgaactctagtaaagaaggctgcacaaggatgcccaagttttcacaattcttggcaggtggtctggtagcattttcatatctatctatatacatttccctctgccacctagcacctacacatttctaaactcactaatctggcaaaattccttgctaccatggaatttcacacaaacagatggtgttgagtaatacatgaggctcattttaatgccactaacaataatgcctcatcctgtcctaattaatgggaagaagctacattgaacagctgtcaaccatgctgctgcattagttatgccgtaagagtgatcaggcgctgcagcccattgtgatgttgccttacaattctgtccacatgaatctgtaccttgcttgattatgcttcaggagagtgtacggaaattagaaaagattgtttaacaataatctggaaatggccttgaattattttttcctcattatttttctcgattaacattctacagaatggtaaggaatcgagacattgctaaaaatcttaaatgactgaaggtatcatagcatcttctgtaaaaaagaaaaaaacttcatattaatttcgattcaaaattttggtcagaaaacaaaaagttgctcttgctttatactttcagggtgaacctggtctcagaggtgaaattggtaaccctggcagagatggtgctcgtgtgagtagaattttgtttgtatgtttcttcgtacttggattttttttttatgtttgaattgagaagttttccaaattagaactacacacactttatttatcaagtttaataaaataatattcctttctctcctgggtctatgactaataatatcattttacatgttccaaaggaaaaattaaatgggatttaacctctttgaaaataatatcctgaattttctaacttccctagtgtcaatgataccaactacaaactatagaccaaaagctttaggtttaatagaatattaaatgattgcttcaagtgataacagagattaaaataaataaataaataagtctcctatgctttaggaagccgggacctctaacaagattctatagttattcaaacctactccctagaaatttatcacccaaagagcagccccaaagattagctgttaatgccatgaagatgccaaagataatcccatgacagtctaattaccttatctcgtatgtcagcctcatgggtcttctaggccacagtcggcctggattcctttattcacctctccttcagagctgaaaactgactgtagcacatctgtaatagtctttcttttgaatcacgtagttctaacagtttcaaacaaggctactcatttgctgctctccagggaattttacaatagcggaaagttcagatctcccaaatttctgacctgctattgacttacacatttccataacctttattactggagtaccctccttctgagagtggcttctaatagtcttgttaattagaaccaaaatacatcagaggccttctagatatccaaccagagtgcagtgaaagtgttcagtcactgtataagcacagaaaaaaagaatgacaaggttcacttttgatgatacggggtgttattaataagacatgtttcctttttggtactagggtgctcctggtgctgtaggtgcccctggtcctgctggagccacaggtgaccgggtaagcatgcattttcactaagccaacagcaatatctaaaatttcccgccttccctagtcccaaagagccccagcaattcatttttatggcttggtataaagcctacttatttaaaaacctagctattgtgatagagcagcaggaaacaaatgctgtgtgtttaaaattacttttcccttcctatagatttgccagctatctgatctatactctaatccctagcatttgttttaaagtctctccatgttgcgcattaacaatatcctaatgcactgaggcttctcaaagccttcaattattaccaaaaaatcaataaaatacatagtgtgcccatttcacattgaactctccacttaaaatagatcttatttattgtattgcaaagattgccacaaatagatcagccccgtgtccatctaaaaattaaaatgtcctcctcctggtattgtaggcactgatttatagtgttttctcaagtgtataacccatactacttaacccccaaaatgaatatagcattaagtaaaaatccacttcattttactctgtgagatgtgcgtcagttatctcttccaaggcaactacagactctgtctgtccaccactgttctctctccctcccagttctttgagcatctatgtcaggcacattaacagattcatctttggtcccattatagggcgaagctggggctgctggtcctgctggtcctgctggtcctcggggaagccctgtaagtaagaacctgggtcattttgtatactcacacctcacaatgtttagacattgatgaacctaggattgataacacatttttaaatcccttctcccacctagggtgaacgtggtgaggtcggtcctgctggccccaatggatttgctggtcctgctgtgagtatcacataatgaagattaatctgaaaacatcctaagttggggagtagagtgggtcggaataccagagctgtaactgtttatttccaacagggtgctgctggtcaacctggtgctaaaggagaaagaggagccaaagggcctaagggtgaaaacggtgttgttggtcccacaggccccgttggagctgctggcccagctgtaagttgaattcactggtggtccacacagcagctacccattagatcttccaattaaatatatatccgtcaagtgcctgctatgcaacagggaatataccagatagaagatggaaaataacggaaggattaacatttgcacactgctttacaaagtataaaagtttcatgaatattgttttattttaattctctgataacctcataagggtggtaatattgaagaacattctgacacagatagtcattttttatttctatattttcttctaagagatgcgggaatgatccacttgaagaaaagagtagcatttacaagggtttgtttgtgatttgactccatctttttgtttgcatttagggtccaaatggtccccccggtcctgctggaagtcgtggtgatggaggcccccctgtgagtatttacaatggactctcgccgcttttcttttttcagaatctattaaggacacttgaaagttttgaaatttttggtaaatttggactaccatgaggaaacttttgagattcaagttcattctattcagagcaattccgatattgatgttaacttgaactcagctggaactcagtgtatgttgctatcagctcacttgaggtaataaccaaggtgggccctaggcagtttaattgtaaagtcggaaaaaatattccttttggcgtttattaatatgccccttcttctgcctgaccatgtccttctcctttgcaggcaatgctatcacaacaattctctagagacccagagctccccaaaaatgaactttactgacttcttctctcactggacagtgctgaattatctaggtcatttgttattcttttgtccatgaacaccattacctattaagtgtccatttccttaccactcagccaggtggtaaagatagttattaatgtatacacattaatgtgtaataatgacatagtgtcttatcttcatacctttacaaccataagataatatgtcagcatttcagaaaggaccatccaaaccttaacgcaaaatatgggcattgcaactggtaatatgctggtaaggaagatgtgtggagaaggagggccttcagggtcctggctaaataatgccctatatgaagctgcctacctcctactccttggtctattcctggtcacatgtactgattttccaaaacaaagaaattcccatcttacccaaattcttggagttgatgttgactgtggaattctaatgtgcttggctcttagggtatgactggtttccctggtgctgctggacggactggtcccccaggaccctctgtaagtaaatcactgtaaacgtgtcttcatttactctagccaaaaggcctggcttctgataggaaactggtaagaaactcttcatgaaaacacatcactaatattcgctattactctcctggtctgaagtcagcttttctgaaccattaaggtatttcatcacaagttatattttataatatcagtttaagaggcttttattcatgtgaacaccagtcccctttcaggggcatggtctttttgaaaaaaaaaaaacaaaaaaacgaacagttttagccacatatcagatatttctatatctaattatcctttatggctaacattctgcctccattgttaaggtataattgttcctgaatttaaaggtggtttggcctctaatttaattctgattcagactctcctgtcaggactcaagaaaatttaattaattaccaaggattaagtcttctggttaaggtttctgggaaaaaaaaatagcaaagatgttgatttcttggaatccttttacaggttcataacagaaaaatcttcattccctgtaggcatttaattaaacctagttgagaagtgtgtgggattcctcaattatgaacaaaacacgtatattggctttctttaaaaaaaaaaaaaaagaagaaaaaagaaaaggcaaagtccttcgaaactcagagtcccattcatttatcattaactcctatcattctacatagttctgattccaatatgccagggtaccagtggcatgacattgtttttcctcatagaaatttgccatagtctctcctccattatttggttggttacagcctcataaaggaagacaggagttgcttctttctgcaagaaagaaggttaaaaactataaatatttcccccaaatggccagggtattattttattgcatcacattgtttgcatcttaagatctagaatctttgctgctctcttccaggcccttggtgattaacagaaaggaaatgaccttgtacatttgctcatagggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctgtaagtgatttccaactcctctttcttaataccttatgctgaattaaaataaagcccctacacaaatcttcaagtggcatctatttgttgatgagtattgcaggctctcaagtagagctcagttgagccaggaaatctgtccagcacacactgaggggctgtggcttccaagatgctcagaaagcacaaaatcgggaagacaataaatgagggaactcagttttatcacaaagcccttaaagctattgaaggcaccttactggtaccaggattagaacagagtcccattgctgtggccatcctactacatattaatcaatctcagtaggctaccaatttcttaaacatacactgtccagtattagtgctactctgaaaggtgcccctattagaccttagaggccaaagtcaaaatttgcttccttttgatatactgattttactgatttcctttttgtttttgttttttgtttttgtttttgtttttttgttttgggatggagttcccctctgttgcccaggctggagtgcagtggcacgatctcagctcactgcgacttccgcctccccagtttaagtaattatcttgtctcagcctcccgagtagctgggactacaggcacacaccatcatgcctagctaatttttgtatttttagtaaagacggggtttcaccatattggtcaggctggtctcgatctcctgacctcaggtgatccacccaccttgacctcctaaagtgctgggattacacatgtgagccaccccacccagcctgatttcctttcctttgtgtatatacccagcagtgtgattgctggatcttatggtagttctatttttagttttttgaggaacccctcctactatgtacaactattatgtatccataacaatttaaatttttttatttgtttccctgcctagaggctataaaaactctatttcaccaccccaagtgtctttataaatctcaaccacatatttttaaatgttgtgccattggtctcaaggatgaatcagatacaaaagtattcatgccaagatgtaaactcaccgtcatcactagagaaaagatatccaaggatatgtcctagtaataggaggtcattagcctttttctaagctgaagacagtttattctcacaatcttcaagccaacctgtgttatcacctagggtcttacccataatactcagtattttttctctatttagggacctcctggcactccaggtcctcagggtcttcttggtgctcctggtattctgggtctccctggctcgagaggtgaacgtggtctaccaggtgttgctggtgctgtggtgagtgcttgacagtattctgactccattaacataagaaaagattttaaaagctgccacttcaaatgtgacagattgatcactgaataacttcacttaagatttttattcatggcattttctttatacagatcacatgtcacttatctaagaagctttaataccaccttacttagacacacactataaagacacagcttaaaattatgcagaatgatttttgttctttccacgcacttaggaatgatacaatctctaattgcgtttactcctctgcaatatgaaatgctggcatcatttatcctgtaggaagaatgaaactctggaagttctgaatcgttccattaaaccttatacgtgacaacaactagaaaccatctcatctccataaaatatatcaactttttgaattcatttcatttgggatactgaatgacacgaggctcactttttacagagcaacatcccgtgattacataaagctggccatctacatgtggagaaggagggcagagatgatactaatgatacttcttaccattgtgtgacccattcacattcaatttaccttcttccttcaaactagaatctcctgagtagggttgttttggaggggaaggttagcattccatcgaataaggggaatgtcattttatcttctctgcctgtttagggtgaacctggtcctcttggcattgccggccctcctggggcccgtggtcctcctggtgctgtgggtagtcctggagtcaacggtgctcctggtgaagctggtcgtgatgtgagtccaacacttggtttgtaaaataaaactgagcaggatttcattgtgtgaaactttatgtcctgagctgaggttctcttcttccaaatttctgaacaagatggtcagcttctccatagtatctacacctagtactgaaaatatgaaaattgcttaggccaaagaatgggcttttcaatagcacactgcaaaactgggcccaagtatttaaaacatctctaaaaaatatctaggttggcaggtttttatccctagttttaacagtctgaaagagggttcgttactgagcactggaagtgatgaagacagagtagctacaacataggggctggtaggcagcagagcctcaccaacagccttaatttgtgtggtgtcttcacagggcaaccctgggaacgatggtcccccaggtcgcgatggtcaacccggacacaaggtcagtacacttttcatctttctctaattcaaaagtgattaaaatgcaacccagattgatgctaagcttcattttgcctttggtagggagagcgcggttaccctggcaatattggtcccgttggtgctgcaggtgcacctggtcctcatggccccgtgggtcctgctggcaaacatggaaaccgtggtgaaactgtaagtttgtgaataccagtccctcagtgcagcattctcgtgggcttcacttctgacttccccacacttggggatggtggaggagtggggaggggtatcttgggcctagctaagttgtgtttttctttttcatttcacagggtccttctggtcctgttggtcctgctggtgctgttggcccaagaggtcctagtgtatgtacatgctgaagatttctttgcaacactaacatttagagagaatcagtccaaaacatctgttaagaaaataaacaatatatcagctagacttaatattttttaaaaatttcagtccatgctgagaattgatacaaataacttgagctattttaaatctcttatgcttgtttgtattaaacaattacaaggatctagccactttacagatagcacaactaaagcagattaccagcagaggtgagagcctagctaaaccattacatgtcctgagttacctttgtaaacgaattaagcagtatttgtggtgaagtgagtgccatttttttaaaacggtaagtcttatccatccttctgtttctttatagggcccacaaggcattcgtggcgataagggagagcccggtgaaaaggggcccagaggtcttcctggcttaaagggacacaatggattgcaaggtctgcctggtatcgctgtaagtaaactgtagccatctcgcacataaactgatcctgaaggccttcagctcagaaggattttcatattttcactgctattgttccagtatagcctatataatatccatttcccattctctggctaactccatctcactcttggaggtaatgctatttcataccaacatgaaaggtgaggattaagggagatagaaatagacatacaataaaatctcctggtaacaatgtccttcaaccccacttaaaataaacataattagaggaatgactaatattgcactgctgaaataggttgtgaaaaaaaaattgaatataatagacataatgggagaaaagagccccactttacattttcaattttctcaatccggagtccatttaactaaagtttcccattcaatttggaaaaaaaaaaaaaatatgtctcttgacatgtgctctgaaagtgtgattttcctcttctgtctttaaagggtcaccatggtgatcaaggtgctcctggctccgtgggtcctgctggtcctagggtaggtggactcaagagaagacagttcatctctgaaatagaggctaaagcgagcagtgagccccaggctgctgctcccttggtgggattcaccagctcacatgtacctggtgtctgtcttccttagggccctgctggtccttctggccctgctggaaaagatggtcgcactggacatcctggtacagttggacctgctggcattcgaggccctcagggtcaccaaggccctgctgtaagtatgatttggggaaataataaagaagatcacggacctaaggaatgttttcttcagactaaaccaagacaactttgacaacccattaaagttagccccatttcaatatatcctctaaaatatctggaaattgtctatatgcaatgggcttgttaagtccatccctgcaagtgtgcctgggggctcgttatttatttatgtgaacttgattattttttactgatgagaacatgcttccgtgtgaagctcaactgaaaatctgctgccatggatgtctctcactgtaacaaaatataaagcctctcctatctcactttcacctttgcagggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggtaggtcaagatgtccagaccagactgacccttctcacaagttgagcttttcaaaattagtttccattgacatttagagtgaaaatgcatttgggtaaagattacattatgtgaaatcacacccaattaatggagcgtcatcttctcccaaccagcacccaacctcatttcccttaaaatgtatttttgcacttttcatagtaataagtaccctgatttgatttttcatggaggaggggagggaaggaactgtctaatcttaaaaatagccaccctcttcctcttaaatatggggtagacaatcaaaaatgttacttatgagagtcagtatctttcattagttattattagaatctgtgttctgctcaatgagaagtttcatgatctgaatgttattttcttaaaaggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccaggtgaggaatcccacaaacacctctccttctgctaaataatattttggtaggactgtttgttaattatctgcattttaatctctgacaaaaatgggcttattaaaaaaagacctgttcctttcctgggttccaattttgtcctaaattgcacattagaagatggattgattggacacatccatgtaattcaaagttattattcaaatttgacttaattggtaatcattgaaaaaactgactaatgtcatttagtgtgaaggagcactggccagctatatgccacactcatacatatgcattttcagaatgtgagcagcttttctgaatttttaatcaaaccttttcaccaactttactgaatgcctactggaattccataaattacaaaatgacagaaaaagaaaaatgtcagaatttctacctcctcattctcttattctaaagaagaagcatatgcaaaaaggattaattgaaacagataacttttttagatgaccttgcctcagtctagtaggtcttatgttcatctaggtaactgatacttcaaagacaagtgaattaagttttctttaaaagtacccttttcctaagcttggatctgagtctactcttcctgagatctttttttttcttttttttttttttcatgtttgactcttagtatctgagtccttctccacttaactggaatttcatcctattttctgtagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctgtaagtaatagtgaaatatgggaatagctttgggaagtgggatggagggggttctaacttagactgcccccaagggggggtctaaaggggggttaaaagaacagaagaatgagagaactaacttatttcataagtaaattcagtttttgtatgtattttatatttatttatttatacgtattaatttcgtacttaaattcagatgataaattcagagtattcttatcagatagtgccttctgaaatgctgaaatgtatactatgtccatgcattgttttttctttagcatgttttttaaatggtaatgtgtgcccagaacttaaaatttcttgagacttcagtggcctaaactataatttatagttatgtgtattttattttacttattagtatggcctacatttaacttttaatgctttttctacaatatgctataaatataagaaaaattaaaattcactaacagcaagactacatacccacccaggtcccgctcccaaagacacacatagagggacatacacacaacaatcctaaaaatgactttgtagagataggtcacttggaatgtgtgttgaaatgttgttggtttttttggtttgtttgtttgtttgttttttgttagactgatagggagcccctcccactaaagacacccttgatactgttatttcaaggatgaacttatttatctgggacagacatcttcagaatgacacatgccaaacagtggttcttattaaatcaaaggttcagatattatcagattcagaaatagtgatgctttgtgtatctattttcttctctttaaacagaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 5 - DNA, 32 bp, Artificial Sequence
  SYNTHETIC NUCLEOTIDE
ggccaagaggaaggaccggtcgagggccaaga

SEQ ID NO: 6 - DNA, 32 bp, Artificial Sequence
  SYNTHETIC NUCLEOTIDE
tcttggccctcgaccggtccttcctcttggcc

SEQ ID NO: 7 - DNA, 30 bp, Artificial Sequence
  SYNTHETIC NUCLEOTIDE
gccaccggtagtgtctaagggcgaagagct

SEQ ID NO: 8 - DNA, 43 bp, Artificial Sequence
  SYNTHETIC NUCLEOTIDE
gccaccggtgcacctccgcccccattaagcttgtgccccagtt

SEQ ID NO: 9 - DNA, 151 bp, Artificial Sequence
  SYNTHETIC NUCLEOTIDE
tcagctttgtggatacgcggactttgttgctgcttgcagtaaccttatgcctagcaacatgccaatctttacaagagcgaaacacctatgcgcctgaaacaacgacgaacgtcattggaatacggatcgttgtacggttagaaatgttctc

SEQ ID NO: 10 - DNA, 34 bp, Artificial Sequence
  SYNTHETIC NUCLEOTIDE
gatcctgtaagaaagggccctaggacattctttc

SEQ ID NO: 11 - DNA, 151 bp, Artificial Sequence; identical to SEQ ID NO: 9
  SYNTHETIC NUCLEOTIDE

SEQ ID NO: 12 - DNA, 34 bp, Artificial Sequence; identical to SEQ ID NO: 10
  SYNTHETIC NUCLEOTIDE

SEQ ID NO: 13 - DNA, 30 bp, Artificial Sequence
  SYNTHETIC NUCLEOTIDE
cgcagatctagtgagcaagggcgaggagct

SEQ ID NO: 14 - DNA, 41 bp, Artificial Sequence
  SYNTHETIC NUCLEOTIDE
cgcagatctccacctccgcccttgtacagctcgtccatgcc